SEQUENCE LISTING (67 sequences)

SEQ ID NO: 1 - protein, 328 aa, Cryptosporidium parvum
MRLSLIIVLLSVIVSAVFSAPAVPLRGTLKDVPVEGSSSSSSSSSSSSSSSSSSSSSTSTVAPANKARTGEDAEGSQDSSGTEASGSQGSEEEGSEDDGQTSAASQPTTPAQSEGATTETIEATPKEECGTSFVMWFGEGTPAATLKCGAYTIVYAPIKDQTDPAPRYISGEVTSVTFEKSDNTVKIKVNGQDFSTLSANSSSPTENGGSAGQASSRSRRSLSEETSEAAATVDLFAFTLDGGKRIEVAVPNVEDASKRDKYSLVADDKPFYTGANSGTTNGVYRLNENGDLVDKDNTVLLKDAGSSAFGLRYIVPSVFAIFAALFVL

SEQ ID NO: 2 - DNA, 987 bp, Cryptosporidium parvum
atgagattgtcgctcattatcgtattactctccgttatagtctccgctgtattctcagccccagccgttccactcagaggaactttaaaggatgttcctgttgagggctcatcatcgtcatcgtcatcgtcatcatcatcatcatcatcatcatcatcatcatcatcatcaacatcaaccgtcgcaccagcaaataaggcaagaactggagaagacgcagaaggcagtcaagattctagtggtactgaagcttctggtagccagggttctgaagaggaaggtagtgaagacgatggccaaactagtgctgcttcccaacccactactccagctcaaagtgaaggcgcaactaccgaaaccatagaagctactccaaaagaagaatgcggcacttcatttgtaatgtggttcggagaaggtaccccagctgcgacattgaagtgtggtgcctacactatcgtctatgcacctataaaagaccaaacagatcccgcaccaagatatatctctggtgaagttacatctgtaacctttgaaaagagtgataatacagttaaaatcaaggttaacggtcaggatttcagcactctctctgctaattcaagtagtccaactgaaaatggcggatctgcgggtcaggcttcatcaagatcaagaagatcactctcagaggaaaccagtgaagctgctgcaaccgtcgatttgtttgcctttacccttgatggtggtaaaagaattgaagtggctgtaccaaacgtcgaagatgcatctaaaagagacaagtacagtttggttgcagacgataaacctttctataccggcgcaaacagcggcactaccaatggtgtctacaggttgaatgagaacggagacttggttgataaggacaacacagttcttttgaaggatgctggttcctctgcttttggactcagatacatcgttccttccgtttttgcaatctttgcagccttattcgtgttgtaa

SEQ ID NO: 3 - protein, 275 aa, Cryptosporidium parvum
DVPVEGSSSSSSSSSSSSSSSSSSSSSTSTVAPANKARTGEDAEGSQDSSGTEASGSQGSEEEGSEDDGQTSAASQPTTPAQSEGATTETIEATPKEECGTSFVMWFGEGTPAATLKCGAYTIVYAPIKDQTDPAPRYISGEVTSVTFEKSDNTVKIKVNGQDFSTLSANSSSPTENGGSAGQASSRSRRSLSEETSEAAATVDLFAFTLDGGKRIEVAVPNVEDASKRDKYSLVADDKPFYTGANSGTTNGVYRLNENGDLVDKDNTVLLKDAG

SEQ ID NO: 4 - protein, 187 aa, Cryptosporidium parvum
DVPVEGSSSSSSSSSSSSSSSSSSSSSTSTVAPANKARTGEDAEGSQDSSGTEASGSQGSEEEGSEDDGQTSAASQPTTPAQSEGATTETIEATPKEECGTSFVMWFGEGTPAATLKCGAYTIVYAPIKDQTDPAPRYISGEVTSVTFEKSDNTVKIKVNGQDFSTLSANSSSPTENGGSAGQASSR

SEQ ID NO: 5 - protein, 12 aa, Cryptosporidium parvum
TGEDAEGSQDSS

SEQ ID NO: 6 - protein, 139 aa, Cryptosporidium parvum
GTEASGSQGSEEEGSEDDGQTSAASQPTTPAQSEGATTETIEATPKEECGTSFVMWFGEGTPAATLKCGAYTIVYAPIKDQTDPAPRYISGEVTSVTFEKSDNTVKIKVNGQDFSTLSANSSSPTENGGSAGQASSRSR

SEQ ID NO: 7 - protein, 83 aa, Cryptosporidium parvum
SEETSEAAATVDLFAFTLDGGKRIEVAVPNVEDASKRDKYSLVADDKPFYTGANSGTTNGVYRLNENGDLVDKDNTVLLKDAG

SEQ ID NO: 8 - DNA, 3066 bp, Cryptosporidium parvum
tctagagtaatagttagagtagatattattcagaacaacgtctaaagtaaaaatgcatatcgtaatcagttacccaaaatattaaaaaaaacgaaaagtcgcaatttagtgcttaggagcttagaccttttttttcccattcagaacaaatccactgcactcagaaattaaaattcaaatcaaaaaaaccttgtattagagggatagtaataaatgcattcgcctcgtcaaaatgctggcacgaattactcggtattcggaaaaaaaaaaatcacctttagtgctattggaatagaaaatctaattaaccacatttcaaaaaaaaataagaacgatgataactttggagttaatatctcattttcgtaggcttgagtgctcaacaacaattatacattagaataaaaataaaagaacatgtaaaagaaccaatatttgtgcattatacgattgagatataattttatgtcttaaataaattaaactttcgcgcaaaaaaattgaaaaaaaaattttgtattacgttctataaaataaaaaagtggtttttcgaattcaatacaaagaataggactcaatataaagtcaaccttgaaattaaattaatataaatttttaagagtagactcgtacgtatgaaatgcttatcgtcttcacatgcatgcaaaaatacgtggactgggtgtatccacataaaaaagcaattaaccacattttacccacacatctgtagcgtcgtcaagtaaaaattgataacaaatttttatacattcggctcgacccttctataggtgataattagtcagtctttaataagtaggcaactaaggacaaaggaagatgagattgtcgctcattatcgtattactctccgttatagtctccgctgtattctcagcctcgtcatcgtcatcatcatcatcatcatcatcatcatcatcatcatcatcaacatcaaccgtcgcaccagcaaataaggcaagaactggagaagacgcagaaggcagtcaagattctagtggtactgaagcttctggtagccagggttctgaagaggaaggtagtgaagacgatggccaaactagtgctgcttcccaacccactactccagctcaaagtgaaggcgcaactaccgaaaccatagaagctactccaaaagaagaatgcggcacttcatttgtaatgtggttcggagaaggtaccccagctgcgacattgaagtgtggtgcctacactatcgtctatgcacctataaaagaccaaacagatcccgcaccaagatatatctctggtgaagttacatctgtaacctttgaaaagagtgataatacagttaaaatcaaggttaacggtcaggatttcagcactctctctgctaattcaagtagtccaactgaaaatggcggatctgcgggtcaggcttcatcaagatcaagaagatcactctcagaggaaaccagtgaagctgctgcaaccgtcgatttgtttgcctttacccttgatggtggtaaaagaattgaagtggctgtaccaaacgtcgaagatgcatctaaaagagacaagtacagtttggttgcagacgataaacctttctataccggcgcaaacagcggcactaccaatggtgtctacaggttgaatgagaacggagacttggttgataaggacaacacagttcttttgaaggatgctggttcctctgcttttggactcagatacatcgttccttccgtttttgcaatctttgcagccttattcgtgttgtaaatttttttcaattaaattttaaaagtttaagagttttaagagtaattgcaatggaaatctttcgtgcgaattcgcattaagggttttgtttattacattgaatcaggacgccagtttttacaatgctgaagaataattaatttttaatttccagaattttcccacgggttccgcaggtttttttcgtatgaatgagttgattatcgtaaacttataagcaaaatagagtaagcaatttggcgagacaattcgaaggattacttagaagattatttggctacttagtttgccttgcgtgatgaagattttaatgtttgagctagatcgagtgaggattaataacagaaaagactaattttttatttaagcgagcttgaaattttttctaagtttcatttttttgtcgggttgtgaatttttctcatgtaccaatggcgtacaatggaaaggcgtgaggtggtgtgtccgaaaagaagtctccgcggttgacaggaatgagtcccatagtgcccagcttgaagaggatgaaggcgattccaaggagtgagtataggcaatataccactttttggatgttgaacggctttcttgctgcgcgctcgatttcaagaaagttcttgtttgcttggattaatatctttactgcattaacgagagcatagctaacgacaagtatgctgaaaattccggcgttattaccgctcatatatagcatgaaaaacgtcatacccatggttttaagtggcagatgagcaatgctccatgccttcttatcaaaattgcaagcttcggaggcattttggtttgtttgtatttctgcgttttcaatcgcacatactgttgggccttggtgttttattctggggtttagcgtcgagttacttgcgcagtgactgttagtattgctactacttgagttgcggtcttgctttgtacgatcgcaaggacttgatcctataagtactcgtttatcaggacttttattaatttttttttcgatagtttttggatttattacccatgcgtcgtacctgctcccaagattcgatcttgtgtcgctttcgagtgtctcttcttgctcatttttcctcattctttggtttgcagtcatttttgagacttgggaatgcttcttcttgtcgggttaaattattttgagcgggaatcttggggggtgagtggttacattaatctgtcgcaaatagttttctaaatgatataatttgtataagttttgactcaaaattaccaattagaatattcaagatcattaaaaaataattgagtctctatatgcat

SEQ ID NO: 9 - protein, 8 aa, Cryptosporidium parvum
DVPVEGSS

SEQ ID NO: 10 - protein, 4 aa, Cryptosporidium parvum
RSRR

SEQ ID NO: 11 - protein, 5 aa, Cryptosporidium parvum
SEETS

SEQ ID NO: 12 - protein, 8 aa, Cryptosporidium parvum
KDAGSSAF

SEQ ID NO: 13 - protein, 7 aa, Cryptosporidium parvum
DDKPFYT

SEQ ID NO: 14 - protein, 6 aa, Cryptosporidium parvum
RSRRSL

SEQ ID NO: 15 - protein, 7 aa, Cryptosporidium parvum
SEETSEA

SEQ ID NO: 16 - protein, 5 aa, Cryptosporidium parvum
ETSEA

SEQ ID NO: 17 - protein, 6 aa, Cryptosporidium parvum
AAATVD

SEQ ID NO: 18 - protein, 4 aa, Cryptosporidium parvum
ATVD

SEQ ID NO: 19 - protein, 14 aa, Cryptosporidium parvum
DVPVEGSSSSSSSS

SEQ ID NO: 20 - protein, 6 aa, Cryptosporidium parvum
TGEDAE

SEQ ID NO: 21 - protein, 10 aa, Cryptosporidium parvum
KSDNTVKIKV

SEQ ID NO: 22 - protein, 34 aa, Cryptosporidium parvum
SEETSEAAATVDLFAFTLDGGKRIEVAVPNVEDA

SEQ ID NO: 23 - protein, 34 aa, Cryptosporidium parvum
ETSEAAATVDLFAFTLDGGKRIEVAVPNVEDASK

SEQ ID NO: 24 - protein, 10 aa, Cryptosporidium parvum
AAATVDLFAF

SEQ ID NO: 25 - protein, 10 aa, Cryptosporidium parvum
ATVDLFAFTL

SEQ ID NO: 26 - protein, 5 aa, Cryptosporidium parvum
AQKRI

SEQ ID NO: 27 - protein, 7 aa, Cryptosporidium parvum
NGDLVDK

SEQ ID NO: 28 - protein, 7 aa, Cryptosporidium parvum
VAVPNVE

SEQ ID NO: 29 - protein, 24 aa, Cryptosporidium parvum
KYSLVADDKPFYTGANSGTTNGVY

SEQ ID NO: 30 - protein, 16 aa, Cryptosporidium parvum
DVPVEGGSSSSSSSSS

SEQ ID NO: 31 - protein, 11 aa, Cryptosporidium parvum
NGGSAGQASSR

SEQ ID NO: 32 - protein, 12 aa, Cryptosporidium parvum
DVPVEGGSSSSS

SEQ ID NO: 33 - protein, 10 aa, Cryptosporidium parvum
KSDNTVKIKV

SEQ ID NO: 34 - protein, 7 aa, Cryptosporidium parvum
VTSVTFE

SEQ ID NO: 35 - protein, 33 aa, Cryptosporidium parvum
GTPAATLKCGAYTIVYAPIKDQTDPAPRYISGE

SEQ ID NO: 36 - protein, 15 aa, Cryptosporidium parvum
TGEDAEGSQDSSGTE

SEQ ID NO: 37 - DNA, 29 bp, Artificial Sequence
  Description of Artificial Sequence Oligonucleotide primers
ctagttccccaccacccaagaccccgtac

SEQ ID NO: 38 - DNA, 21 bp, Artificial Sequence
  Description of Artificial Sequence Oligonucleotide primers
aaggggtggtgggttctgggg

SEQ ID NO: 39 - DNA, 19 bp, Artificial Sequence
  Description of Artificial Sequence Oligonucleotide primers
ctagttccccaccacccaa

SEQ ID NO: 40 - DNA, 32 bp, Artificial Sequence
  Description of Artificial Sequence Oligonucleotide primers
gatcgttgagcaagttcagcctggtaagtgca

SEQ ID NO: 41 - DNA, 21 bp, Artificial Sequence
  Description of Artificial Sequence Oligonucleotide primers
gatcgttgagcaagttcagcc

SEQ ID NO: 42 - DNA, 24 bp, Artificial Sequence
  Description of Artificial Sequence Oligonucleotide primers
cttaccaggctgaacttgctcaac

SEQ ID NO: 43 - DNA, 20 bp, Artificial Sequence
  Description of Artificial Sequence Oligonucleotide primers
tgcacttaccaggctgaact

SEQ ID NO: 44 - DNA, 17 bp, Artificial Sequence
  Description of Artificial Sequence Oligonucleotide primers
gchgthcchaaygthga

SEQ ID NO: 45 - DNA, 20 bp, Artificial Sequence
  Description of Artificial Sequence Oligonucleotide primers
gtrtaraawggyttrtcrtc

SEQ ID NO: 46 - DNA, 22 bp, Artificial Sequence
  Description of Artificial Sequence Oligonucleotide primers
ctcttttagatgcatcttcgac

SEQ ID NO: 47 - DNA, 21 bp, Artificial Sequence
  Description of Artificial Sequence Oligonucleotide primers
gtcgagatgcatctaaaagag

SEQ ID NO: 48 - DNA, 21 bp, Artificial Sequence
  Description of Artificial Sequence Oligonucleotide primers
gctgctgcaaccgtcgatttg

SEQ ID NO: 49 - DNA, 24 bp, Artificial Sequence
  Description of Artificial Sequence Oligonucleotide primers
ggcggatctgcgggtcaggcttca

SEQ ID NO: 50 - DNA, 23 bp, Artificial Sequence
  Description of Artificial Sequence Oligonucleotide primers
tcttggtgcgggatctgtttggt

SEQ ID NO: 51 - DNA, 21 bp, Artificial Sequence
  Description of Artificial Sequence Oligonucleotide primers
cagggttttcccagtcacgac

SEQ ID NO: 52 - DNA, 22 bp, Artificial Sequence
  Description of Artificial Sequence Oligonucleotide primers
tcacacaggaaacagctatgac

SEQ ID NO: 53 - DNA, 18 bp, Artificial Sequence
  Description of Artificial Sequence Oligonucleotide primers
taggcttgagtgctcaac

SEQ ID NO: 54 - DNA, 18 bp, Artificial Sequence
  Description of Artificial Sequence Oligonucleotide primers
gggctgagaatacagcgg

SEQ ID NO: 55 - DNA, 19 bp, Artificial Sequence
  Description of Artificial Sequence Oligonucleotide primers
ccagaagcttcagtaccac

SEQ ID NO: 56 - DNA, 17 bp, Artificial Sequence
  Description of Artificial Sequence Oligonucleotide primers
ttggtgcgggatctgtt

SEQ ID NO: 57 - DNA, 19 bp, Artificial Sequence
  Description of Artificial Sequence Oligonucleotide primers
tcccacgggttccgcaggt

SEQ ID NO: 58 - DNA, 20 bp, Artificial Sequence
  Description of Artificial Sequence Oligonucleotide primers
tcctcactcgatctagctca

SEQ ID NO: 59 - DNA, 20 bp, Artificial Sequence
  Description of Artificial Sequence Oligonucleotide primers
gtacaaagcaagaccgcaac

SEQ ID NO: 60 - DNA, 26 bp, Artificial Sequence
  Description of Artificial Sequence Oligonucleotide primers
gatgttcctgttgagggctcatcatc

SEQ ID NO: 61 - DNA, 26 bp, Artificial Sequence
  Description of Artificial Sequence Oligonucleotide primers
tcttgatcttgatgaagcctgacccg

SEQ ID NO: 62 - DNA, 19 bp, Artificial Sequence
  Description of Artificial Sequence Oligonucleotide primers
tcagaggaaaccagtgaag

SEQ ID NO: 63 - DNA, 22 bp, Artificial Sequence
  Description of Artificial Sequence Oligonucleotide primers
accagcatccttcaaaagaact

SEQ ID NO: 64 - protein, 81 aa, Cryptosporidium parvum
ETSEAAATVDLFAFTLDGGKRIEVAVPNVEDASKRDKYSLVADDKPFYTGANSGTTNGVYRLNENGDLVDKDNTVLLKDAG

SEQ ID NO: 65 - protein, 77 aa, Cryptosporidium parvum
AAATVDLFAFTLDGGKRIEVAVPNVEDASKRDKYSLVADDKPFYTGANSGTTNGVYRLNENGDLVDKDNTVLLKDAG

SEQ ID NO: 66 - protein, 75 aa, Cryptosporidium parvum
ATVDLFAFTLDGGKRIEVAVPNVEDASKRDKYSLVADDKPFYTGANSGTTNGVYRLNENGDLVDKDNTVLLKDAG

SEQ ID NO: 67 - protein, 6 aa, Cryptosporidium parvum
AVPNVE

(19) United States
(12) Patent Application Publication
(10) Pub. No.: US 2003/0185836 A1
(43) Pub. Date: Oct. 2, 2003

(54) Cryptosporidium sporozoite antigens

(76) Inventors: Gerhard Winter, Eastwood (AU); Martin Basil Slade, East Ryde (AU); Keith Leslie Williams, Avalon (AU); Andrew Arthur Gooley, Turramurra (AU)

Correspondence Address: MORGAN LEWIS & BOCKIUS LLP, 1111 PENNSYLVANIA AVENUE NW, WASHINGTON, DC, 20004, US

(21) Appl. No.: 10/148,687

(22) PCT Filed: Dec. 1, 2000
(86) PCT No.: PCT/AU00/01492

(30) Foreign Application Priority Data
Dec. 1, 1999 (AU) PQ 4400

(51) Int. Cl. A61K 39/00; A61K 39/38; A61K 39/002

(52) U.S. Cl. 424/184.1

(57) ABSTRACT

Antigenic polypeptides and peptides from Cryptosporidium, and nucleic acid molecules encoding same, are dis-closed which have potential for use in a protective vaccine preparation. A preferred polypeptide has the amino acid sequence, (a).

```
                10 20 30 40
MRLSLIIVLL SVIVSAVFSA PAVPLRGTLK DVPVEGSSSS
                50 60 70 80
SSSSSSSSSS SSSSSSSTST VAPANKARTG EDAEGSQDSS
                90 100 110 120
GTEASGSQGS EEEGSEDDGQ TSAASQPTTP AQSEGATTET
              130 140 150 160
IEATPKEECG TSFVMWFGEG TPAATLKCGA YTIVYAPIKD
              170 180 190 200
QTDPAPRYIS GEVTSVTFEK SDNTVKIKVN GQDFSTLSAR
              210 220 230 240
SSSPTENGGS AGQASSRSRR SLSEETSEAA ATVDLFAFTL
              250 260 270 280
DGGKRIEVAV PNVEDASKRD KYSLVADDKP FYTGANSGTT
              290 300 310 320
NGVYRLNENG DLVDKDNTVL
```

### FIELD OF THE INVENTION

The present invention relates to the identification of target molecules for the treatment of cryptosporidiosis. In particular, the invention relates to the discovery of a molecule on the Cryptosporidium sporozorite cell-surface that represents a candidate molecule for vaccine development.

### BACKGROUND TO THE INVENTION

Cryptosporidium is a protozoan parasite causing a serious diarrhoea which may be life threatening in immunocompromised people. Cryptosporidium also infects a wide range of vertebrates including birds, reptiles and fish. Cryptosporidium meleagridis from birds can infect humans (73, 74). The study by Sreter et al was undertaken in order to characterize Cryptosporidium meleagridis isolated from a turkey in Hungary and to compare the morphologies, host specificities, organ locations, and small-subunit RNA (SSU rRNA) gene sequences of this organism and other Cryptosporidium species. The phenotypic differences between C. meleagridis and Cryptosporidium parvum Hungarian calf isolate (zoonotic genotype) oocysts were small, although they were statistically significant. Oocysts of C. meleagridis were successfully passaged in turkeys and were transmitted from turkeys to immunosuppressed mice and from mice to chickens. The location of C. meleagridis was the small intestine, like the location of C. parvum. A comparison of sequence data for the variable region of the SSU rRNA gene of C. meleagridis isolated from turkeys with other Cryptosporidium sequence data in the GenBank database revealed that the Hungarian C. meleagridis sequence is identical to a C. meleag,ridis sequence recently described for a North Carolina isolate. Thus, C. meleagridis is a distinct species that occurs worldwide and has a broad host range, like the C. parvum zoonotic strain (also called the calf or bovine strain) and Cryptosporidium felis. Because birds are susceptible to C. meleagridis and to some zoonotic strains of C. parvum, these animals may play an active role in contamination of surface waters not only with Cryptosporidium baileyi.

It is likely that only strains from mammals can infect humans (49, 50). The parasite is generally transmitted by the faecal-oral route. Person to person transmission is likely to be the major cause of persistence in the community rather than water supplies (65, 64). However, contamination of water supplies with the chlorine-resistant oocysts has caused large outbreaks of cryptosporidosis in the USA and England (63). At present, at least two distinct genotypes have been shown to cause disease in humans (66, 67). Genotype 1 was found in cryptosporidosis in humans, but not in animals and so is thought to be only transmitted between humans. Genotype 2 was isolated from both humans and calves and so is likely to have a zoonotic transmission cycle. The details of the sequences suggest genotype 2 could be further divided into subtypes.

The protozoan parasite Cryptosporidium parvum is increasingly recognised as an important cause of diarrhoea, particularly in the aged individuals and infants. C. parvum is also a common intestinal infection in immunocompromised patients (eg AIDS, cancer patients, recipients of transplants) causing a chronic, watery diarrhoea and weight loss which may develop into a life threatening condition (2). There is no effective treatment available (3).

In immunocompetent hosts the disease is controlled immunologically as indicated by resistance to reinfection following recovery from a self limiting infection (4, 5, 6, 7, 8, 9). This concept is supported by persons who rapidly cleared C. parvum infections on termination of immunosuppressive therapy (10). Both T lymphocyte-mediated immunity and humoral response are major effector mechanisms for the resolution of infections in the immunocompetent host (11). Antibodies have been shown to control C. parvum infections in both mice and humans (7, 12, 13, 14, 15). In particular, hyperimmunised bovine colostrum, is effective in treating C. parvum infections in SCID mice (16). In contrast, monoclonal antibodies reduced, but did not cure persistent C. parvum infections in immunodeficent scid mice (54).

Of particular interest for the management of immunosuppressed AIDS patients is reports that oral administration of antibodies can be effective in treating C. parvum infections. Tzipori et al (17, 18) reported that four immunodeficient patients with cryptosporidiosis recovered and remained free of diarrhoea within 3-5 days after treatment via a nasogastric tube with immune colostrum from cows hyperimmunised with oocysts/sporozoites of C. parvum. IgG1 and IgA were the most active fractions of hyperimmune colostrum for the treatment of experimental infections in neonatal mice (19). Ungar et al (53) also reported the successful use of anti-Cryptosporidium bovine colostrum to treat an AIDS patient who remained free of diarrhoea and oocysts for three months. A double-blind, controlled pilot study reported a significant improvement in two out of three HIV patients given a continuous naso-astric infusion of hyperimmune anti-C. parvum bovine colostrum (21). It is notable that reports of successful treatment have used large volumes (50-480 ml daily) of bovine colostrum, presumably to maintain a high anti-C. parvum antibody concentration in the lumen of the gut. Both bovine colostrum and serum, neutralised sporozoites faster than either individual or pooled anti-Cryptosporidium monoclonal antibodies (22). The present inventors know of no reports of monoclonal antibodies being successfully used in the treatment of human cryptosporidiosis, perhaps because of the large amounts required; 84 ug/day/mouse (23) is roughly equivalent to a 0.2 g dose/day for a human. High specificity polyclonal antiserum is likely to be both cheaper and more effective for use as an oral treatment of cryptosporidiosis because it reacts with multiple epitopes.

Immunisation with whole inactivated C. parvum oocyst has been shown to partially protect calves against challenge with 10⁴C. parvum oocysts (48). However, there was no indication of which of the many components of the oocysts conferred protection.

The primary targets for immune intervention are the stages of the C. parvum life cycle that infect epithelial cells, the sporozoites and merozoites. When C. Parvum oocysts are ingested, sporozoites excyst and parasitise the epithelial layers of the gastrointestinal or respiratory tract (reviewed 24). Intracellular multiplication involves several morphological forms including inerozoites, which infect new host cells. Sexual stages lead to the development of oocysts which sporulate in situ. Most oocysts are shed in the faeces, but it is thought that some release sporozoites that repeat the infective cycle. The sporozorite is the only invasive form that can be prepared in substantial quantities. However, the merozoites closely resemble sporozoites and infect new host cells by a process morphologically similar to that of sporozoites. Indeed, sporozoites and merozoites have some common epitopes (27, 28, 31, 39) and are both recognised by antibodies from hyperimmune bovine colostrum (6). Thus it may be possible to get protection against both sporozoites and merozoites using antibodies to some sporozoite antigens. Few antigenic differences have been detected among C. parvum isolates from different patients, countries or animal hosts (25, 26, 24).

Targets for immune intervention include surface proteins involved in motility and the attachment of the sporozorite to the host cell. Several sporozorite surface proteins have been described, but comparison is complicated because many appear to have similar molecular weights (within lab to lab experimental variation). The P23 sporozoite antigen is present on the cell surface (27, 28) and is shed into trails left by migrating sporozoites, causing it to be proposed as a potential adhesin (29, 30). P23 is highly immunogenic and usually recognised by convalescent sera from humans and animals (20, 27). Daily oral treatment with monoclonal antibodies against P23 reduced the parasite load of experimentally infected mice (23, 52). Riggs et al (15) have identified additional protein antigens that are recognised by neutralising monoclonal antibodies. Antibody 17.41 caused significant neutralisation of 25 times the ID50 dose of sporozoites for mice and it recognised surface antigens with apparent molecular masses-of 28, 55 and 98 kDa. Another monoclonal antibody recognising a 15 kDa, highly immunogenic glycoprotein found in sporozoites and merozoites has been shown to reduce oocyst production by 67% when given orally to infected mice (31). Several further surface proteins have been identified and partially functionally characterised (32, 33). A metallo-dependent cysteine proteinase associated with the sporozorite surface has been described which may be important in the infection process (34). Whilst this molecule may not be immunogenic in natural infections, passively administered specific antibodies raised against the isolated proteins may confer protection. Although the definition of neutralisation antigens using monoclonal antibodies has been a considerable step forward, they only reduced parasite loads in vivo in mouse models when used as a mixture of antibodies (23) and then not as effectively as anti-Cryptosporidium bovine colostrum (19).

Production of good polyclonal antisera requires a substantial supply of purified antigens, as does direct vaccination of patients. C. parvum is an obligate intracellular parasite that grows poorly in both tissue culture cells and the chorioallantoic membrane of chicken embryos (1) so the best source is the faeces of infected animals which may contain up to 10⁷ oocysts/g. However, faeces are inappropriate for the preparation of purified bulk antigens for therapeutic use. The best alternative is to use molecular biology techniques to produce pure recombinant proteins. Consequently, there have been several reported attempts to clone the respective genes of neutralising antigens. These attempts rely in all cases on the indirect screening of expression libraries with specific antibody probes. Using this approach a cDNA was expressed encoding an epitope shared by 15 and 60 kDa proteins (35). Jet injection of a recombinant plasmid encoding this cDNA section into sheep resulted in high titer colostrum reactive with the sporozorite surface (36). However, the interpretation of this research (35, 36) is complicated by frame shift in the original DNA sequence that gave an incorrect amino acid sequence for half the gene (51). Sagodira et al. (69) and Iochmann et al. (70) cloned and expressed the gene of Jenkins et al (35) coding for the 15 and 60 kDa antigens from sporozoites and vaccinated animals with some degree of success. It should be noted however that the two antigens for which the genes code bear no significant homology with that of this invention even when the frame shift in the initial DNA sequence that gave an incorrect amino acid sequence for half the antigen was taken into consideration (51). The cloning of a different DNA sequence for a 13 kDa protein was reported by Mead et al. (37). This sequence shows no homology to the gene obtained by Jenkins et al. (35). The relation of both sequences to the previously identified surface glycoprotein gp5 protein remains unclear. A further report claims the cloning of a gene encoding neutralization-sensitive epitopes (38). A consensus open reading frame was identified after screening of a cDNA library with monoclonal antibodies directed against a 23 kDa protein. Serum raised against a synthetic peptide derived from the consensus sequence was reactive with p23. However, in none of these studies have any C. parvum surface proteins been characterised directly.

The present inventors have now cloned a gene from Cryptosporidium that encodes polypeptides and peptides suitable for use as antigens. It should be noted that the gene described in this disclosure has no sequence identity or similarity with those described in the above studies.

### DISCLOSURE OF THE INVENTION

In a first aspect, the present invention provides an isolated nucleic acid molecule encoding a Cryptosporidium polypeptide comprising the amino acid sequence:

MRLSLIIVLLSVIVSAVFSAPAVPLRGTLKDVPVEGSSSSSSSSSSSSSS SSSSSSSTSTVAPANKARTGEDAEGSQDSSGTEASGSQGSEEEGSEDDGQ TSAASQPTTPAQSEGATTETIEATPKEECGTSFVMWFGEGTPAATLKCGA YTIVYAPIKDQTDPAPRYISGEVTSVTFEKSDNTVKIKVNGQDFSTLSAN SSSPTENGGSAGQASSRSRRSLSEETSEAAATVDLFAFTLDGGKRIEVAV PNVEDASKRDKYSLVADDKPFYTGANSGTTNGVYRLNENGDLVDKDNTVL LKDAGSSAFGLRYIVPSVFAIFAALFVL (SEQ ID NO: 1), a functionally equivalent sequence thereof, or part thereof having at least five amino acids.

Preferably, the isolated nucleic acid molecule encodes a polypeptide with the amino acid sequence shown as SEQ ID NO: 1.

Preferably, the isolated nucleic acid molecule comprises a nucleotide sequence substantially as shown as SEQ ID NO: 2, or a functionally equivalent nucleotide sequence thereof, or a sequence which hybridises to the nucleotide sequence of SEQ ID NO: 2, or a sequence which shows at least 60% homology with the nucleotide sequence of SEQ ID NO: 2. More preferably, the nucleic acid molecule has at least 80% homology with the nucleotide sequence of SEQ ID NO: 2 and most preferably the nucleic acid molecule has at least 90% homology with that sequence.

As is stated above the present invention includes nucleic acid molecules which hybridis to the sequence shown in SEQ ID NO: 1. Preferably such hybridisation occurs at, or between, low and high stringency conditions. In general terms, low stringency conditions can be defined as 3×SCC at about ambient temperature to 65° C., and high stringency conditions as 0.1×SSC at about 65° C. SSC is the abbreviation of a buffer of 0.15 M NaCl, 0.015 M trisodium citrate. Three×SSC is three times as strong as SSC and so on.

As will be recognised by those skilled in the art, recombinant expression vectors suitable for transformation of a host cell (such as a suitable bacterial, yeast, insect or mammalian host cell) including the nucleic acid molecule of the present invention operably linked to a regulatory sequence can be prepared. When host cells are transformed with such an expression vector the transformed cells can be used for preparing the polypeptides which preferably have amino acid sequences substantially as shown as SEQ ID NO: 1.

As used herein the term “functionally equivalent nucleotide sequence” is intended to cover minor variations in the encoding nucleotide sequence which, due to degeneracy in the DNA code, does not result in the molecule encoding a polypeptide having substantially different biological or lowered antigenic activity from the native polypeptide. This may be achieved by various changes in the sequence, such as insertions, deletions and substitutions, either conservative or non-conservative, where such changes do not substantially adversely alter the biological or antigenic activity of the encoded polypeptides.

In a second aspect, the present invention provides an isolated polypeptide from Cryptosporidium comprising the following sequence:

DVPVEGSSSSSSSSSSSSSSSSSSSSSTSTVAPANKARTGEDAEGSQDSS GTEASGSQGSEEEGSEDDGQTSAASQPTTPAQSEGATTETIEATPKEECG TSFVMWFGEGTPAATLKCGAYTIVYAPIKDQTDPAPRYISGEVTSVTFEK SDNTVKIKVNGQDFSTLSANSSSPTENGGSAGQASSRSRRSL SEETSEAAATVDLFAFTLDGGKRIEVAVPNVEDASKRDKYSLVADDKPFYT GANSGTTNGVYRLNENGDLVDKDNTVLLKDAG (SEQ ID NO: 3), or a functionally equivalent sequence thereof, or part thereof having at least five amino acids.

Preferably, the polypeptide has the amino acid sequence shown as SEQ ID NO: 1.

In a further preferred form of the second aspect of the present invention, the polypeptide has at least the following amino acids modified by a reducing terminal alpha-GalNAc (indicated by underline):

DVPVEGSSSSSSSSSSSSSSSSSSSSSTSTVAPANKRTGEDAEGSQDSS GTEASGSQGSEEEGSEDDGQTSAASQPTTPAQSEGATTETIEATPKEECG TSFVMWFGEGTPAATLKCGAYTIVYAPIKDQTDPAPRYISGEVTSVTFEK SDNTVKIKVNGQDFSTLSANSSSPTENGGSAGQASSRSRRSL SEETSEAAATVDLFAFTLDGGKRIEVAVPNVEDASKRDKYSLVADDKPFYT GANSGTTNGVYRLNENGDLVDKDNTVLLKDAG (SEQ ID NO: 3), or a functionally equivalent sequence thereof, or part thereof having at least five amino acids.

As used herein the term “functionally equivalent amino acid sequence” is intended to cover minor variations in the amino acid sequences described which results in a polypeptide having relative activity which is not substantially less than that of the corresponding native polypeptide. Preferably, a polypeptide having an altered amino acid sequence from the sequence shown as SEQ ID NO: 1 has substantially the same or greater activity or antigenicity than that of the native polypeptide. This may be achieved by various changes in the sequence, such as insertions, deletions and substitutions.

It will be appreciated that “conservative” changes which would not be expected to adversely change the activity or antigenicity of the polypeptide or polypeptides according to the present invention are also included within the scope of the present invention. Conservative substitutions include polypeptide analogs wherein at least one amino acid residue in the polypeptide has been replaced by a different amino acid. Such substitutions are made in accordance with the following Table 1, which substitutions may be determined by routine experimentation to provide modified structural and functional properties of a synthesised polypeptide molecule while maintaining biological and antigenic activity.

TABLE 1

| Original Residue | Exemplary Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

Alternatively, another group of substitutions are those in which at least one amino acid residue in the polypeptide has been removed and replaced with a different residue in its place according to Table 2 below. Alternative conservative substitutions are defined herein as exchanges within one of the following five groups set out in Table 2.

TABLE 2

| Group No | Description |
|---|---|
| 1 | small aliphatic non-polar or slightly polar residues: |
| | Ala, Ser, Thr (Pro, Gly) |
| 2 | polar negatively charged residues and their amides: |
| | Asp, Asn, Glu, Gin |
| 3 | polar positively charged residues: |
| | His, Arg, Lys |
| 4 | large aliphatic non-polar residues: |
| | Met, Leu, Ile, Val, (Cys) |
| 5 | large aromatic residues: |
| | Phe, Tyr, Trp |

The three amino acid residues in parentheses above have special roles in protein architecture. Gly is the only residue lacking any side chain and thus imparts flexibility to the chain. This however tends to promote the formation of secondary structure other than α-helical. Pro, because of its unusual geometry, tightly constrains the chain and generally tends to promote μ-turn-like structures, although in some cases Cys can be capable of participating in disulfide bond formation which is important in protein folding. Note also that Tyr, because of its hydrogen bonding potential, has significant kinship with Ser and Thr.

Conservative amino acid substitutions according to the present invention, as described above, are known to the art and would be expected to maintain biological and structural properties of the polypeptide after amino acid substitution. Most deletions and substitutions according to the present invention are those which do not produce radical changes in the characteristics of the protein or polypeptide molecules.

In a third aspect, the present invention provides a vaccine preparation against Cryptosporidium containing one or more polypeptides comprising an amino acid sequence selected from:

(SEQ ID NO: 4)

DVPVEGSSSSSSSSSSSSSSSSSSSSSTSTVAPANKARTGEDAEGSQDSS
GTEASGSQGSEEEGSEDDGQTSAASQPTTPAQSEGATTETIEATPKEECG
TSFVMWFGEGTPAATLKCGAYTIVYAPIKDQTDPAPRYISGEVTSVTFEK
SDNTVIKIKVNGQDFSTLSANSSSPTENGGSAGQASSR;

or functionally equivalent sequences thereof, or parts thereof having at least five amino acids.

In a fourth aspect, the present invention provides a vaccine preparation against Cryptosporidium containing one or more polypeptides comprising an amino acid sequence selected from:

(SEQ ID NO: 5)

TGEDAEGSQDSS;

(SEQ ID NO: 6)

GTEASGSQGSEEEGSEDDGQTSAASQPTTPAQSEGATTETIEATPKEECG
TSFVMWFGEGTPAATLKCGAYTIVYAPIKDQTDPAPRYISGEVTSVTFEK
SDNTVKTKVNGQDFSTLSANSSSPTENGGSAGQASSRSR;

or functionally equivalent sequences thereof, or parts thereof having at least five amino acids.

In a fifth aspect, the present invention provides a vaccine preparation against Cryptosporidium containing one or more polypeptides comprising an amino acid sequence selected from:

(SEQ ID NO: 7)

SEETSEAAATVDLFAFTLDGGKRIEVAVPNVEDASKRDKYSLVADDKPFYT
GANSGTTNGVYRLNENGDLVDKDNTVLLKDAG;

or functionally equivalent sequences thereof, or parts thereof having at least five amino acids.

It will be appreciated that the vaccines according to the present invention may further comprise suitable diluents and adjuvants and the like known to the art.

In a sixth aspect, the present invention provides a method of immunising a subject against Cryptosporidium, the method comprising providing a vaccine preparation according to the third, fourth or fifth aspects of the present invention to the subject such that an immune response is generated in the subject against Cryptosporidium.

The method is applicable to animals including humans.

The vaccine preparation may be provided to the subject by any of the common administration routes used in the art (e.g. intramuscular, subcutaneous and nasal administration).

The present inventors have identified, cloned and sequenced a new gene encoding a family of major surface glycoprotein(s) found on the surface of the sporozoite, the stage of the Cryptosporidium life cycle that initiates the infection of the intestinal wall. The gene sequence is not present in publicly accessible data-bases and bears no homology to previously described genes.

The S60 gene consists of a 987 bp open reading frame shown in FIG. 1 (SEQ ID NO: 2) and shown with flanking sequences in FIG. 2 (SEQ ID NO: 8).

The precursor to the S60 gene encodes a 328 amino acid sequence:

(SEQ ID NO: 1)

MRLSLIIVLLSVIVSAVFSAPAVPLRGTLKDVPVEGSSSSSSSSSSSSSS
SSSSSSSTSTVAPANYARTGEDAEGSQDSSGTEASGSQGSEEEGSEDDGQ
TSAASQPTTPAQSEGATTETIEATPKEECGTSFVMWFGEGTPAATLKCGA
YTIVYAPIKDQTDPAPRYISGEVTSVTFEKSDNTVKIKVNGQDFSTLSAN
SSSPTENGGSAGQASSRSRRSLSEETSEAAATVDLFAFTLDGGKRIEVAV
PNVEDASKRDKYSLVADDKPFYTGANSGTTNGVYRLNENGDLVDKDNTVL
LKDAGSSAYGLRY1VPSVFAIFAALFVL.

It is apparent that the hydrophobic leader sequence is cleaved from the precursor molecule co-translationally as is the case with the majority of proteins that are destined for export from eukaryote cells. The position of that cleavage is likely to be in the vicinity of the aspartic acid residue 31, giving the mature S60 molecule the amino acid sequence:

(SEQ ID NO: 3)

DVPVEGSSSSSSSSSSSSSSSSSSSSSTSTVAPAN1KARTGEDAEGSQDS
SGTEASGSQGSEEEGSEDDGQTSAASQPTTPAQSEGATTETIEATPKEEG
GTSFVMWFGEGTPAATLKCGAYTIVYAPIKDQTDPNPRYISGEVTSVTFE
KSDNTVKIKVNGQDFSTLSANSSSPTENGGSAGQASSRSRRSLSEETSEA
AATVDLFAFTLDGGKRIEVAVPNVEDASKRDKYSLVADDKPFYTGANSGT
TNGVYRLNENGDLVDKDNTVLLKDAG.

There is clear evidence that the S60 gene product is processed into two glycopeptides S15 and S45. The N-terminus of protein S45 starts at aspartic acid residue 31, with the sequence DVPVEGSS (SEQ ID NO: 9). All peptides in S45 lie on the N-terminal side of the predicted cleavage sequence RSRR (residues 217-220; SEQ ID NO: 10) in S60. Protein S45 contains peptides spanning the following mature protein sequence (residues 31-222):

(SEQ ID NO: 4)

SDNTVKIKVNGQDFSTLSANSSSPTENGGSAGQASSRDVPVEGSSSSSSS
SSSSSSSSSSSSSSTSTVAPANKARTGEDAEGSQDSSGTEASGSQGSEEE
GSEDDGQTSAASQPTTPAQSEGATTETIEATPKEECGTSFVMWFGEGTPA
ATLKCGAYTIVYAPIKDQTDPAPRYISGEVTSVTFEKSDNTVKIKVNGQD
FSTLSANSSSPTENGGSAGQASSR.

All peptides in S15 lie on the C-terminal side of the predicted cleavage sequence RSRR (residues 217-220) in S60. Protein S15 has a N-terminal sequence starting at residue 223 (ie SEETS; SEQ ID NO: 11). It is predicted that S15 is cleaved in the vicinity of the glycine residue (305) in the sequence KDAGSSAF (SEQ ID NO: 12) with the addition of a glycosyl phosphatidyl inositol anchor to account for the amphipathic properties of the protein. Protein S15 contains peptides spanning the following mature protein sequence (residues 222-305):

(SEQ ID NO: 7)

SEETSEAAATVDLFAFTLDGGKRIEVAVPNVEDASKRDKYSLVADDKPFY
TGANSGTTNGVYRLNENGDLVDKDNTVLLKDAG-linked to GPI
anchor.

The present inventors have cloned gene segment fragments for S60, S45 and S15 in E. coli expression vectors for the production of polypeptides for the immunisation of animals. In doing so it is realised that the addition of carbohdydrates to the recombinant molecule may be important for the appropriate immunogenicity of the antigen. As such, it may be preferable to express the antigen in alternate expression systems such as insect cells infected with recombinant baculovirus, fungal cells such as the yeasts Saccharomyces cerevisiae, Schizosaccharomyces pombe or Pichia pastoris. Whilst it is realised that these systems may not add the same sugars to the recombinant molecule as is found in Cryptosporidium, the presence of sugars is likely to assist the molecule to attain a higher degree of immunogenicity than the non-glycosylated molecule.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In order that the present invention may be more clearly understood, preferred forms will be described with reference to the following example and accompanying figures.

### BRIEF DESCRIPTION OF THE ACCOMPANYING FIGURES

FIG. 1 shows the DNA sequence of the S60 gene.

FIG. 2 shows the DNA sequence of the S60 gene and flanking regions with key features.

FIG. 3 shows the translated amino acid sequence of the S60 gene.

FIG. 4 shows processing of the S60 gene and S45 and S15 proteolytic fragments.

FIG. 5 provides a list of N-terminal sequences of peptides according to the present invention.

FIG. 6 shows oligonucleotides used for cloning and sequencing the S60 gene.

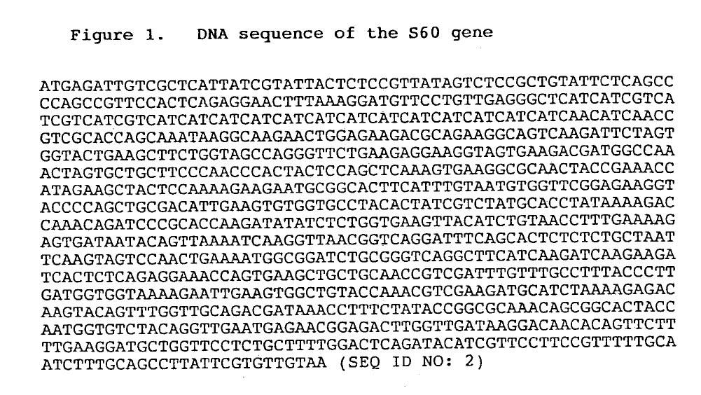

### EXAMPLE

### Experimental Methods

1. Isolation of C. parvum oocysts

Faecal samples positive for Cryptosporidium parvum were obtained from naturally infected calves. The faecal samples were diluted in 2 volumes of water and centrifuged at 5000×rpm (3000×g) for 10 mins. The liquid layer was discarded, the pellet resuspended in water and the procedure repeated. Fatty materials were removed by suspending the pellet in 2 volumes ice cold 1% (w/v) NaHCO₃ solution, adding 1/3 volume of ice cold ether and centrifuging at 5000×rpm (3000×g) for 10 minutes. The supernatant containing a plug of fat was discarded and the pellet resuspended in ice cold 1% (w/v) NaHCO₃ solution and passed through a layer of prewetted nonabsorbent cotton wool. The resulting eluant was then re-extracted with ether. The final pellet was resuspended in 40 ml of ice cold 55% sucrose solution. Then 10 ml of ice cold water was slowly layered on to the surface, ensuring 2 layers were formed and centrifuged at 4000 rpm for 20 minutes. Oocysts were collected from the surface interface and the sucrose flotation step repeated until no visible contaminating material could be detected. Purified oocysts were surface sterilised with ice cold 70% (v/v) ethanol for 30 min, washed once in phosphate buffered saline pH 7.5 (PBS; Oxoid) and stored in PBS at 4° C.

For further purification of oocysts and the isolation of sporozoites cells were labelled with anti-oocyst monoclonal antibody CRY26 (39) and anti-mouse IgG directed antibodies coupled to magnetic beads (Myltenyi, Bergisch-Gladbach, Germany). Oocysts labelled with magnetic beads were separated from non-magnetic contaminants using a Myltenyi cell sorter consisting of a steel wool column placed in a magnetic field. For sporozoite isolation oocysts were excysted in 0.75% taurocholate for 30 minutes at 37° C. Non-magnetic sporozoites were separated from labelled oocysts, oocyst walls and debris using the Myltenyi cell sorter and washed once in Hanks buffered salts solution (IHBSS; pH 7.5).

2. Biotinylation

Freshly isolated, viable sporozoites were washed in PBS and resuspended at 1×10⁸ cells/100 ul in prewarmed (37° C.) PBS. 100 mM NHS-LC-Biotin (Pierce) in DMSO was added to a final concentration of 1 mM and incubated for 10 minutes at 37° C. The reaction was stopped by the addition of 10 mM Tris/HCl pH 8.0. Cells were then washed three times in TBS (10 ml Tris/140 mN NaCl, pH 8.0) and resuspended in PBS.

3. Hybridoma Production

Monoclonal antibody CRY41 was obtained from N. Pererva (39). Briefly, BALB/c mice were immunized by intraperitoneal injection with 1-10×10⁶ excysted C. parvum oocysts emulsified in Freund's complete adjuvant. Two injections followed at 3-4 weeks intervals with the same antigen mixture in Freund's incomplete adjuvant (FIA). A further 5 booster injections were carried out with gamma-irradiated (190 Gy) oocysts in FIA with the final injection given intravenously 2 days prior to fusion. Initial screening of hybridoma supernatants was by an enzyme-linked immunosorbent assay (ELISA). Excysted oocyst mixture were homogenised in PBS (4×10⁵ cells/ml) and 50 ul applied to ELISA plate wells. Plates were air dried overnight and blocked with 2% (w/v) BSA in TBS (10 mM Tris-buffered saline, pH 7.5) for 1 h at 37° C. Hybridoma supernatants (100 ml/well) were added and plates incubated for 1 h at 37° C., then washed 3 times with TBS and further incubated with 100 ml/-well horseradish peroxidase conjugated sheep anti-mouse immunoglobulin diluted in 2% BSA/TBS. Plates were washed twice and developed in 100 ml/ well of substrate solution containing 0.4 mg/ml phenylenediamine in citrate buffer (pH 5.0) and 0.009% H₂O₂. The reaction was stopped by the addition of 2 M sulphuric acid (50 ml, well) and optical densities measured at 450 nm with an automated ELISA plate reader (Dynatech MR7000). Positive supernatants were reexamined by an indirect immunofluorescence assay as described below for reactivity against a sporozorite surface antigen. The clone CRY41 was selected and further characterised by Western Blot analysis. CRY41 was determined to be IgM isotype using a commercial assay (Sigma).

4. Fluorescence Microscopy

Freshly excysted oocysts or purified sporozoites were fixed in 2% formaldehyde/0.05% glutaraldehyde/PBS for 20 minutes and washed three times in PBS. In experiments using biotinylated sporozoites, cells were labelled prior to fixation. Approximately 10⁵ sporozoites were applied to each well of polylysine precoated microscope slides. Sporozoites were allowed to settle for 15 minutes, the supernatant aspirated and the sporozoites were overlaid with 1% (w/v) BSAIPBS for 15 minutes. For indirect immunofluorescence, sporozoites were incubated with culture supernatant of hybridoma cell line CRY41 for 30 minutes. Wells were washed three times with PBS and then overlaid with fluorescein-conjugated goat anti-mouse IgG antibodies diluted 1:50 in 1% BSA/PBS. After 15 minutes of incubation wells were washed three times in PBS and mounted in 50% Glycerol/PBS containing an anti-oxidant DAPCO (Hoechst AG, Germany). For the detection of biotin residues, labelled sporozoites were incubated for 30 minutes with fluorescein-conjugated streptavidin diluted 1:100 in PBS. Wells were then washed and mounted as described above.

For detection of sporozoite trails, sporozoites suspended in HBSS pH 7.5 were applied on polylysine-coated slides and incubated for 15 minutes at 37° C. prior to fixation. Wells were washed three times in PBS and processed as described above.

5. Cell Lysis and Triton X-114 Subfractionation

Excysted oocysts or purified sporozoites were resuspended to 1×10⁹ cells/ml in ice-cold TBS (10 mM Tris, pH 8.0, 140 mM NaCl) containing a protease inhibitor mixture of 50 μM leupeptin (Sigma),.10 μM E-64 (Sigma), 1 mM phenylmethylsulfonylfluoride (Sigma). Precondensed Triton X-114 (Ref 61) was added to a final concentration of 2% (v/v) and left on ice for 30 minutes. Lysates were then centrifuged (100,000×g for 1 h at 4° C.) and the supernatant warmed for 3 minutes to 37° C. to induce phase separation. The water phase and the membrane protein enriched detergent phase were separated by centrifugation and each phase reextracted once. The phases were adjusted to the original volume and proteins precipitated by adding 1/10 volume of ice cold 100% (w/v) trichloroacetic acid. After 30 minutes precipitates were pelleted by centrifugation and washed twice in 70% (v/v) ice-cold ethanol and once with acetone. Pellets were then air dried and redissolved in 1-D or 2-D sample buffer for electrophoretic analysis.

6. SDS-PAGE

C. parvum cell protein samples were diluted with 0.25 volume of sample buffer (62 mM Tris, 2.0% SDS, 10% (w/v) glycerine, 5% (v/v) betta-mercaptoethanol, 0.001% bromophenol blue, pH 6.8) for 5 minutes at 100° C. and electrophoresed using the Mini Protean II gel apparatus (Bio-Rad) and a discontinuous buffer system of Laemmli (56). Proteins were electrophoresed until the dye marker reached the bottom of the gel and then subjected to Western blot analysis.

7. Two Dimensional gel Electrophoresis (2-D PAGE)

TCA precipitated C. parvum cell protein fractions equivalent to 2 mg total cell protein were solubilised in 100 ul sample buffer containing 8 M Urea, 4% (w/v) CHAPS, 2% Pharmalyte 3-10 (v/v, Pharmacia), 2% (w/v) dithiothreitol (DTT) and insoluble material removed by centrifugation. Nonlinear Immobiline DryStrips (pH 3-10; 18 cm; Pharmacia) were used for the first-dimensional isoelectric focusing (IEF). Each strip was placed in 2 ml tissue culture pipettes and rehydrated overnight in 8M Urea, 4% Chaps, 2% DTT, 0.5% Pharmalyte 3-10 prior to sample application. IEF was carried out using a Pharmacia Multiphor II with a Consort 5000 V power supply. Temperature was controlled at 200° C. Samples were applied cathodically in sample cups (Pharmacia) and focused with discontinuous voltage steps of 300V for 5 h, 1000 V for 5 h, 2500 V for 5 and 5000 V to a total of 250 kVh. The IEF strips were immediately processed for the second dimensional SDS-PAGE run. Strips were equilibrated for 10 minutes in 50 mM Tris, 6 M UREA, 30% (v/v) glycerol, 2% (v/v) SDS, pH 6.8 and DTT (2% w/v). In a second 10 minute equilibration the DDT was replaced with iodo acetamide (2.5%, w/v). The second dimension SDS-PAGE gradient (9-16% T) were 1.5 mm thick and prepared with 0.12 M Tris/acetate, pH 6 as gel buffer and piperazine diaciylamide at 2.5% C as cross-linker. The anode buffer consisted of 45 mM Tris-acetate, pH 6.6 with 0.1% (w/v) SDS and cathode buffer was 80 mM Tricine-Tris, pH 7.1 with 0.1% (w/v) SDS and 0.001% (w/v) bromophenol blue. Strips were placed on top of 9-16% T SDS-PAGE gradient gels and embedded in molten 0.5% (w/v) agarose in cathode buffer. Gels were run with 20 mV constant voltage at 10° C. until the dye front reached the bottom of the gel: The separated proteins were either stained with silver diamine or blotted onto PVDF membranes for western analysis.

8. Western Blotting

Proteins separated by SDS-PAGE were electrophoretically transferred to nitrocellulose or polyvinylidene difluoride membranes (Bio-Rad) using a discontinuous buffer system (57) in a semi-dry electroblotting system. One dimensional gels were electroblotted for 1 hour at 12 V and 2-D gels for 3 hours at 300 mA. Transferred proteins were either stained with 0.5% (w/v) amido black or transiently stained with 0.1% (w/v) Ponceau S in 1% (v/v) acetic acid for 5 minutes prior to detection with antibodies, streptavidin reagent or lectins.

For antibody staining, membranes were blocked for 1 h with 5% milk powder in PBS. Membranes were incubated for 1 hour with CRY41 culture supernatant, washed thrice with PBS and then incubated for 1 hour with alkaline phosphatase conjugated goat anti-mouse IgG (Promega) antibody diluted 1:100 in PBS. All steps were performed at room temperature.

For detection of biotinylated proteins blots were blocked for 1 hour with 1% BSA/PBS and then incubated for 3 hours with alkaline phosphatase conjugated ExtrAvidin (Sigma) diluted 1:10 000 in PBS. Blots were developed in 1 M diethanolarnine, pH 9.8, 1 mm MgCl₂ containing 0.5 mM 5-bromo-4-chloro-3-indolyl phosphate and 0.5 mM nitroblue tetrazolium.

For lectin analysis, membranes were blocked with 1% (w/v) BSA and then incubated for 1 hour with biotin-conjugated lectins (Sigma) in PBS, washed and incubated for a further 3 hours with ExtrAvidin-alkaline phosphatase (Sigma) in PBS. When the lectin ConA was used, all buffers were supplemented with 1 mM MgCl₂, 1 mM CaCl₂.

9. Peptide digestion

After 2-D PAGE and transfer to PVDF, membranes were stained with amido black and individual spots excised and digested with endoproteinase Glu-C (Boehringer Mannheim) as described by Fernandez et al (58) with minor modifications. Prior to the enzyme treatment, spots were reduced and alkylated using tributyl phosphilne and acrylamide in an automated version of the procedure described by Brune (59, 60). Spots were then placed into an 0.5 ml Eppendorf tube and incubated for 24 hours at 37° C. in 50 μl 100 mM Tris, pH 8.0, 1% hydrogenated Triton X-100 containing 1 μg endoproteinase Glu-C. Spots were sonicated for 5 minutes and the supernatant transferred to a fresh vial and stored at −200° C. The digestion of the spots was repeated followed by sonication. Spots were then washed once with 100 μl 0.1% TFA and the combined supernatants (200 μl) were stored at −20° C. Seventy (70) μl of the pooled digest was loaded onto a Pharmacia Sephasil C8 column (2.1 mm×100 mm) and peptides separated by reversed-phase chromatography using the SMART HPLC system. The chromatography program consisted of a linear gradient of 100% buffer A (0.15% TFA) to 60% buffer B (0.1% TFA, 85% v/v acetonitrile) over 25 minutes and then 60% buffer B to 100% buffer B over 5 minutes at a flow rate of 100 μl/min. Peptides were detected at 214 nm. Peptide chroinatograms were compared with a blank area of the PVDF membrane digested and separated as described above.

10. Protein Sequencing

The N-terminal sequence was obtained for individual protein spots identified on PVDF membranes after staining with amido black or purified peptides. Amino terminal sequencing was conducted on a Model G1000A (Hewlett-Packard, Calif.) sequenator using program 3.1 chemistry for Edman degradation (68). A PTH-amino acid standard for Ser-alpha-GalNAc and Thr-alpha-GalNAc was generated from sequence analysis of a synthetic peptide containing a Ser-alpha-GalNAc and Thr-alpha-GalNAc.

11. Preparation of genomic C. parvum DNA

Genomic DNA was prepared from 5×10⁸ sporozoites (stored as a frozen pellet at −70° C.) resuspended in 500 μl 10 mM Tris, pH 8.0, 0.1 M EDTA, 5% SDS with 20 μg/ml pancreatic RNAse (Sigma) and incubated for 2 hours at 37° C. One hundred (100) μg proteinase K was added and proteins digested at 50° C. for 2 hours. Samples were then extracted twice with phenol and precipitated with 2 volumes of ethanol. DNA was further purified over glassmilk (Bresa-Clean DNA purification kit, Bresatec Ltd., Australia) and stored in TE buffer (10 mM Tris, pH 8.0, 0.1 mM EDTA) at 4° C.

12. Directional Genomic Walking

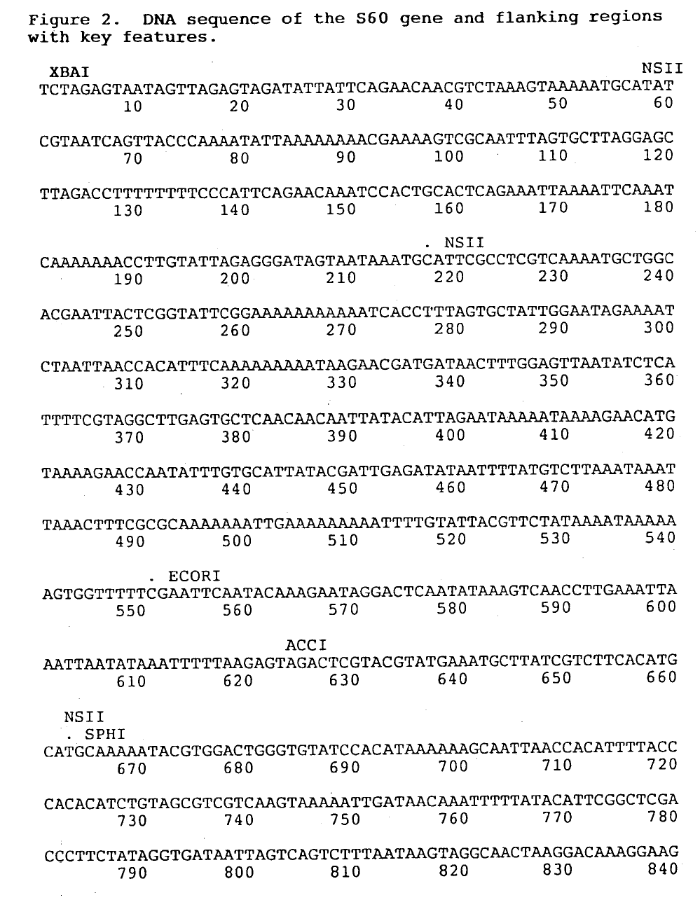

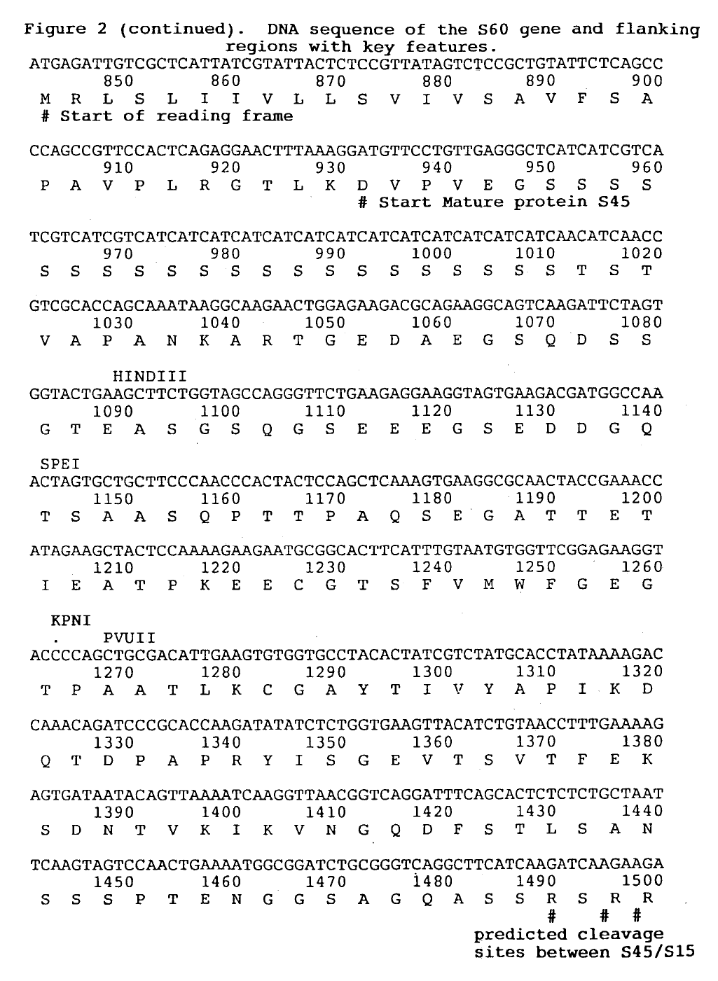

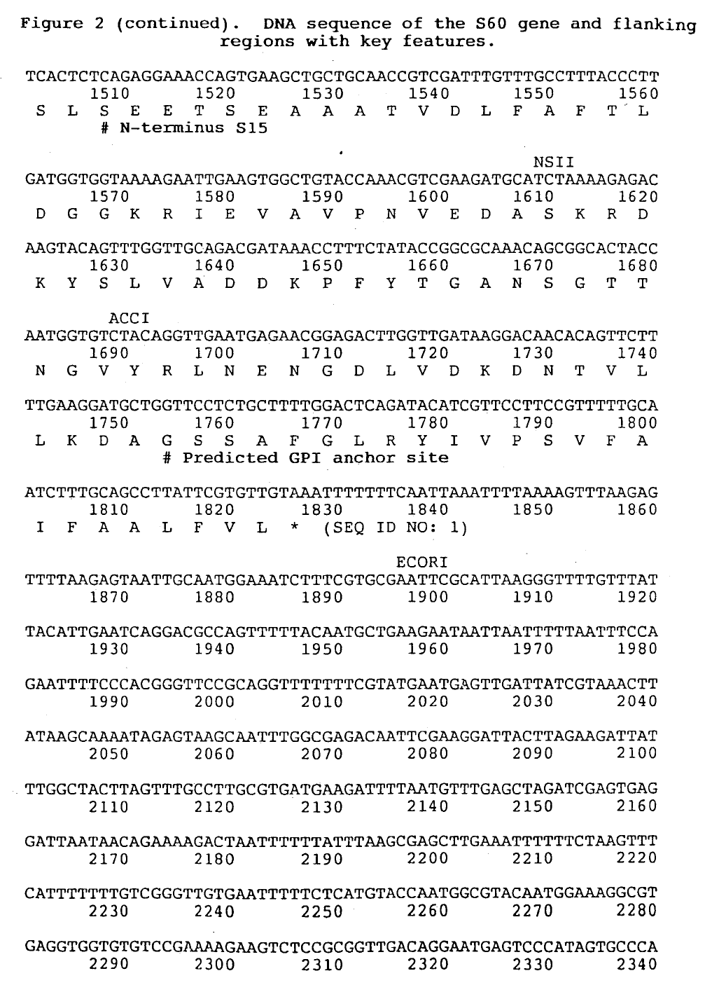

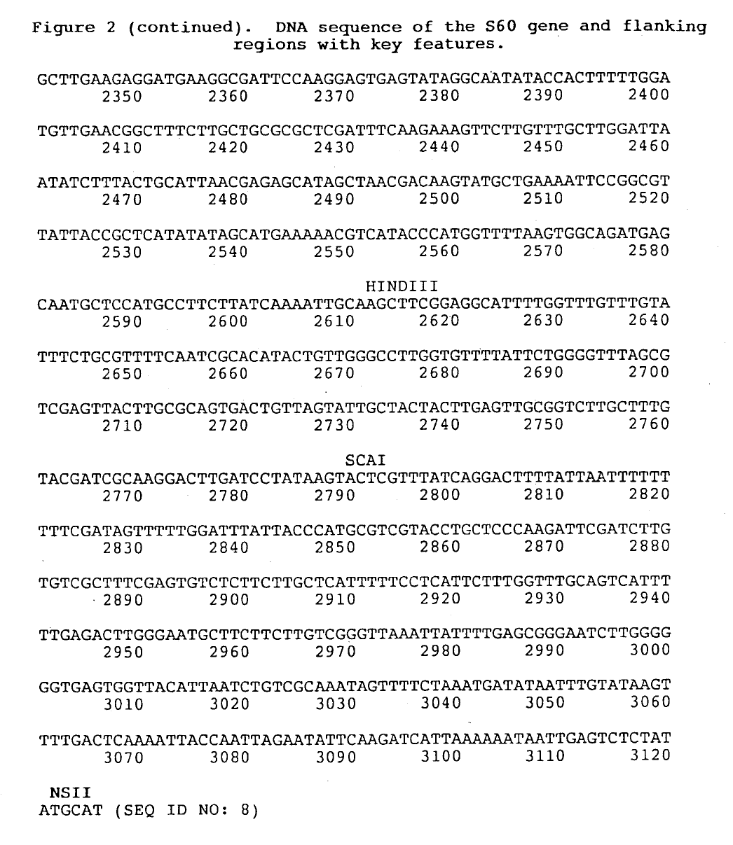

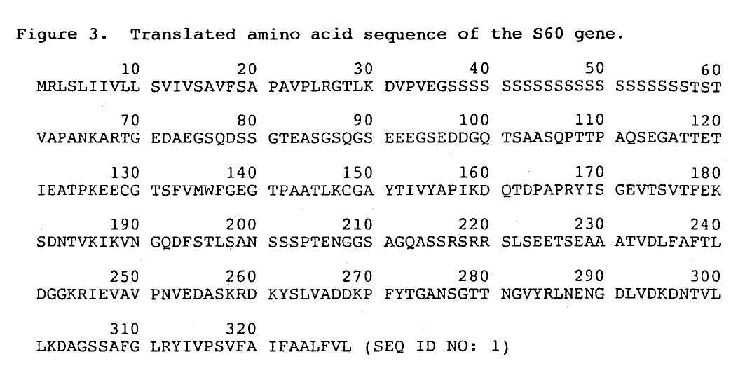

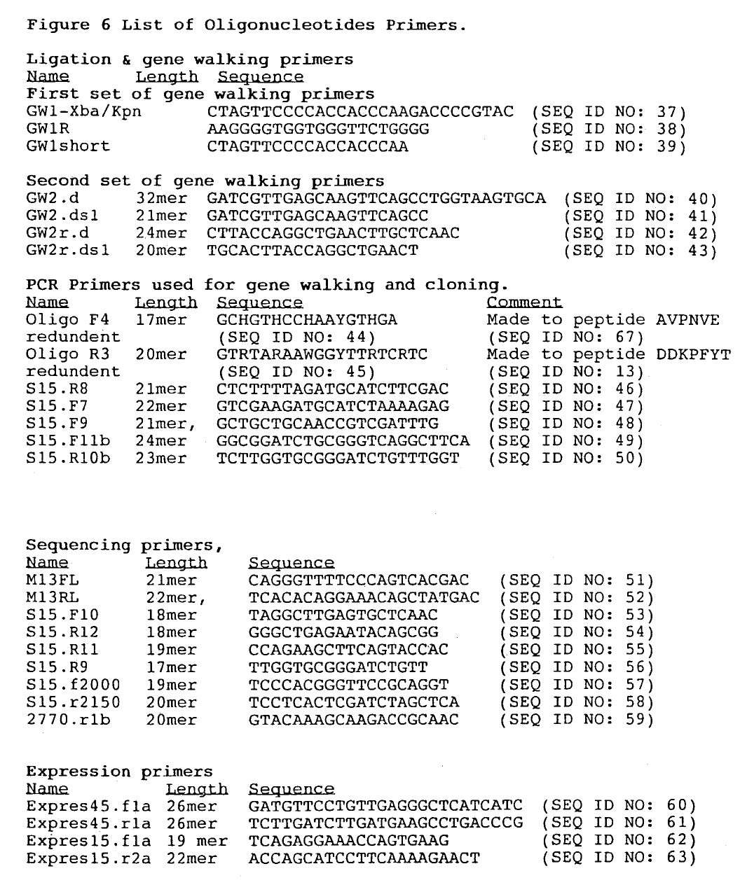

A 73 bp gene fragment corresponding to position 1585-1658 of the DNA sequence shown in FIG. 2 was amplified by PCR using the degenerate oligonucleotides S15F4 and S15R3 (FIG. 6). S15F4 was designed from the amino acid sequence AVPNVE (SEQ ID NO: 67) in peptide fraction 17 obtained from the S15 protein spot and reverse primer S15FR3 was designed from the amino acid sequence DDKPFYT (SEQ ID NO: 13) in peptide fraction 21 from the S15 protein spot. The PCR reaction was performed using the Taq DNA polymerase (Boehringer) in a 25 μl reaction mixture containing 2 pM of forward and reverse primer, 1 μl of template genomic DNA, 0.1 mM deoxynucleotide triphosphates (dNTPs), 1 U Taq DNA polymerase and 5 mM MgCl₂. The PCR program was 30 cycles of 94° C. for 30 sec, 47° C. for 30 sec and 72° C. and ended with a single step of 72° C. for 5 min.

A directional genomic walking strategy based on a procedure described by Morris et al. (44) was used to isolate the S60 gene. Genomic DNA (200 ng in 20 μl) was digested overnight with 10 U of various restriction enzymes and ligated to linkers used to construct linker libraries. Two sets of linkers were designed (FIG. 6) which contained unique 3′ and 5′ prime over hangs compatible with the ends created by the restriction digests of genomic DNA. Primer set 1 was compatible with XBAI and KpnI restriction enzymes, while primer set 2 was compatible with BamHI, BglII, BclI, NsiI and PstI. 750 pmol of the linkers for top and bottom strands in 50 μl TE buffer were denatured for 1 minute at 94° C. and reannealed at 50° C. for 30 min. Approximately 10 ng digested genomic DNA and 15 pMoles linker were ligated in a 10 μl reaction mixture containing 5 U of T4 ligase (Boehringer) overnight at 16° C. The linker library was finally diluted to 50 Al with TE buffer and stored at −20° C.

Genomic sequences were amplified by PCR using specific walking primers derived from known DNA sequences and generic linker primers which were derived from the top or bottom strand of each of the linkers used (listed in FIG. 6). PCR was performed using the Taq DNA polymerase or the Expand Taq system from (Boehringer). Standard 25 μl reaction mixtures contained 1 μl of DNA linker library and 20 pmol each of specific walking primer and linker primer.

13. Cloning and Sequencing of PCR Products

PCR products were purified using the Wizard PCR Preps Kit (Promega) and cloned in TOP10 one shot E. coli using a TOPO TA Cloning Kit (Invitrogen). Plasmids were isolated using the Plasmid-Wizard kit (Promega) and used as template DNAs in BigDye cycle sequencing (Applied Biosystems Inc.). Sequence primers are listed in FIG. 6.

14. Expression in E. coli

Peptides S60, S15 and S45 have been expressed as recombinant proteins in E. coli. Initially the whole gene was amplified using the Expand PCR kit (Boehringer) from genomic DNA using the sequencing primers S15.F10 and S15.r2150 that bind in the DNA flanking the S60 gene. The resulting 1.8 Kb PCR fragment containing the gene was gel purified using a QIAquick gel extraction kit (Qiagene) and used as substrate for amplification of the gene sequences. The DNA sequences encoding the mature peptides of S60, S15 and S45 were amplified by PCR techniques using the expression oligonucleotides shown in FIG. 6. The S45 expression primers amplify DNA encoding the protein sequence from residue 31 to 219, ie from the N-terminus of the mature S45 protein to the second arginine residue in RSRRSL (SEQ ID NO: 14). The S15 expression primers amplify the DNA encoding residues 223-305, ie from the N-terminus of the S15 protein to the predicted position for the addition of a GPI anchor to the native protein. The S60 gene DNA encoding residues 31-305 was amplified using the Expres45.f1a primer that binds at the N-terminus of S45 and with primer Expres15.r2a that binds the gene at the predicted position for the addition of a GPI anchor to the native protein.

The PCR fragments were inserted into pBAD TOPO TA expression vector (Invitrogen) and transformed into TOP10 one shot E. coli (Invitrogen). Expression was induced by the addition of 2 mM L-arabinose to the culture medium for 4 hours. The recombinant protein S60 was detected by a monoclonal antibody to a HIS tag (Invitrogen) and had apparent molecular weight of approximately 45 kDa. The difference is likely to be due to glycosylation and other post-translational modifications in the native protein (since the molecular weights predicted from the amino acid sequence are 28.2, 18.7 and 9.1 kDa for the S60, S45 and S15 peptide respectively) and its strange amino acid composition of 29% S +T amino acids and the consequent negative charge that this imposes upon the molecule.

15. Production of Polyclonal Anti-rS60 Antisera

Recombinant S60 protein was purified by metal affinity chromatography using the Xpress purification system (Invitrogen). Fractions containing rS60 protein were separated by SDS-PAGE and blotted onto nitrocellulose membrane. The rS60 band was ground to very fine pieces and suspended in Freund's complete adjuvant. BALB/c mice were injected intraperitoneally with 1-2 μg of antigen followed by three booster injections with membrane-bound antigen in incomplete Freund's adjuvant.

16. Western Blotting

Proteins separated by SDS-PAGE were electrophoretically transferred to nitrocellulose or polyvinylidene difluoride membranes (PVDF; Bio-Rad) using a discontinuous buffer system (Khyse-Anderson, 1984). One-dimensional gels were blotted for 1 h at 12 V and two-dimensional gels for 3 h at 300 mA. Transferred proteins were either stained with 0.5% (w/v) amido black or transiently stained with 0.1% (w/v) Ponceau S in 1% (v/v) acetic acid for 5 min prior to detection with antibodies, streptavidin reagent or lectins;

For antibody staining, membranes were blocked for 1 h with 5% (w/v) milk powder in PBS. Membranes were then incubated for 1 h with CRY41 culture supernatant and then 1 h with 1:100 alkaline phosphatase conjugated anti-mouse IgG (Promega) in 5% (w/v) milk/PBS. For detection of biotinylated proteins, membranes were blocked with 5% (w/v) milk/PBS and then incubated for 3 h with alkaline phosphatase conjugated ExtrAvidin (Sigma) diluted 1:10,000 in PBS.

For lectin analysis, membranes blocked with 1% (w/v) BSA in PBS were incubated for 1 h with biotin-conjugated lectins (Sigma) in BSA/PBS and a further 3 h with 1:10,000 alkaline-phosphatase-conjugated ExtrAvidin in PBS. When Canavalia ensiformis lectin (Con A) was used, all buffers were supplemented with 1 mM MgCl₂ and 1 mM CaCl₂. Membranes were developed in 1 M diethanolamine, pH 9.8, containing 1 mM MgCl₂, 0.5 mM 5-bromo-4-chloro-3-indolyl phosphate and 0.5 mM nitroblue tetrazolium.

### Results

This work arises from extensive work characterising the sporozoite antigens by surface labelling and monoclonal antibodies. In outline, the bulk amounts of Cryptosporidium oocysts were purified from faeces from intensively reared calves. It proved possible to select highly purified, undamaged sporozoites by antibody labelling oocyst prior to excystation, the excysted sporozoites pass through an antibody binding column while oocysts walls, whole oocysts and any contaminants (eg. yeast cells) are retained.

The surface proteins of Cryptosporidium sporozoites were identified and characterised by surface labelling with biotin, with lectins ConA and H. pomatia lectin and with monoclonal antibody CRY41 produced at Macquarie University. On SDS-PAGE electrophoresis and Western blotting, the biotinylated surface proteins stained with avidin gave prominent bands with apparent molecular masses of 15 and 45 kDa and multiple bands in the regions 25-29 and >60 kDa. The 15 and 45 kDa bands were identified by labelling with H. pomatia lectin and not with ConA (as were other bands). Crucially, the monoclonal antibody CRY41 reacted with both the 15 kDa protein band and in immunofluorescence microscopy stained the surface of intact sporozoites and trails left behind the migrating sporozoites.

The proteins were purified by two dimensional (2-D) electrophoresis. The proteins from whole oocysts were extracted and the membrane proteins separated by Triton X114 phase separation. In this procedure, membrane proteins are found in the detergent phase while soluble proteins partition into the aqueous phase. 2-D gels of the detergent phase revealed the most prominent protein spots had acidic pIs close to pH 4 and apparent molecular masses of 15 kDa and 45 kDa, with a smear of protein from the 45 kDa spot to around 60 kDa. Probing western blots with lectins and CRY41 indicated that the major spots on the 2-D gel were the same proteins as seen on 1-D SDS-PAGE.

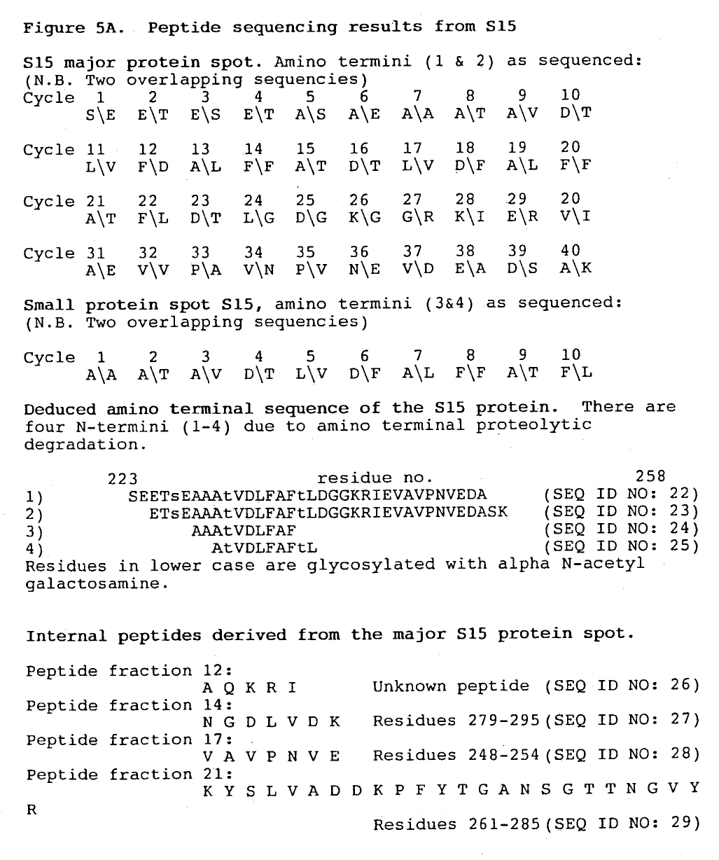

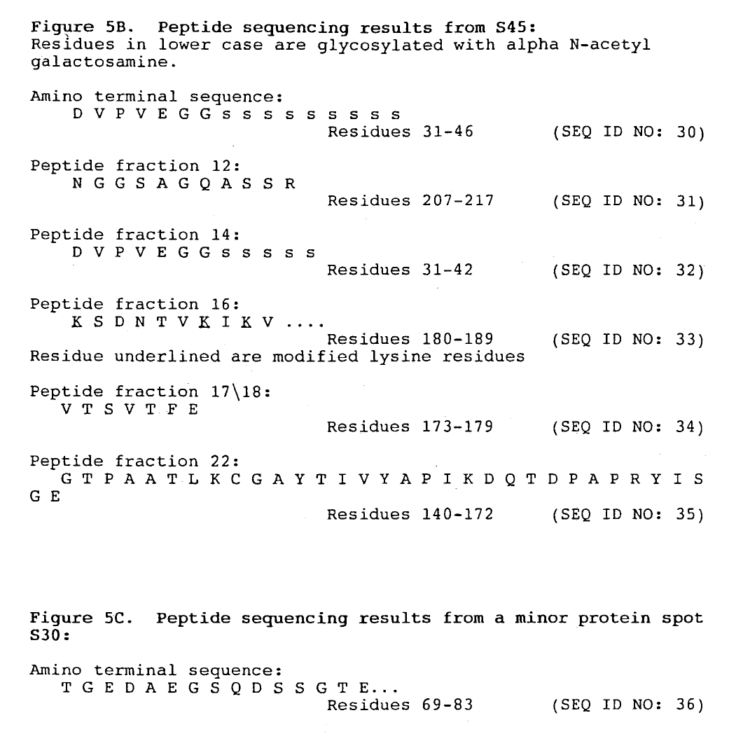

The protein spots were characterised by protein sequencing. The 2-D gels were blotted on to PVDF membranes and the protein spots analysed by Edman degradation chemistry using an aqueous phase transfer to HPLC which allows the detection of glycosylated amino acids. The N-terminus of the main 15 kDa spot gave two overlapping sequences in equimolar amounts (FIG. 5A), which later in the light of the DNA sequence could be interpreted as starting at SEETSEA (SEQ ID NO: 15) and ETSEA (SEQ ID NO: 16). In addition, sequencing of a minor spot (below the main S15 spot) gave also gave two overlapping N-terminal sequences (AAATVD; SEQ ID NO: 17 and ATVD; SEQ ID NO: 18) which occur slightly further into the S15 gene. Thus S15 has at least four different N- termini (see FIG. 5A). This “ragged end” is likely to be due to the action of a amino peptidase previously reported from sporozorite membranes (62).

The 45 kDa protein gave clear N-terminal sequence DVPVEGSSSSSSSS. (FIG. 5B; SEQ ID NO: 19) containing a long stretch of serine residues glycosylated with single residues of alpha N-acetyl galactosamine. The first serine residue was only partially glycosylated, but the following residues were completely glycosylated. The detection of alpha N-acetyl galactosamine is consistent with the lectin staining reactions of the protein. Note, a sample from the protein smear at around 60 kDa detected a single N-terminal sequence identical to the 45 kDa protein spot, indicating it contained the 45 kDa protein. A likely explanation for the smear is that some of the 45 kDa protein was poorly solubilised in the second dimension of the 2D gel. However, the presence of some S60 protein that has not been cleaved at residues 218-222 between S45 and S15) can not be excluded.

S30, a minor spot on the 2-D gel between the major S15 and S45 spots, appeared to be a proteolytic degradation product of S45. The N-terminal sequence of S30 (FIG. 5C) of TGEDAE (SEQ ID NO: 20) indicates portion of S45 was cleaved at the C-terminal side of arginine 68, removing the heavily glycosylated poly serine sequence at the start of S45.

The protein spots also gave internal peptide sequences. The 15 and 45 kDa protein spots on PVDF were digested with the protease GluC and the peptide fragments separated on reversed phase HPLC. The two proteins gave different peptide peaks, indicating the 45 kDa protein does not contain peptides from the 15 kDa protein. The N-terminal sequences of the purified peptides are listed in FIG. 5A & 5B. Peptide 16 from S45 gave unusual chromatograph peaks for residues 180, 186 and 188 in the sequence KSDNTVKIKV (SEQ ID NO: 21). These residues eluted close to where PTH tryptophan elutes, but were thought likely to be modified lysine residues. This was later confirmed by the DNA sequence encoding lysine at these positions.

Peptide sequences were used to design PCR oligos to clone S15. Redundant oligonucleotide (forward) primer S15F4 was designed from the amino acid sequence AVPNWE (SEQ ID NO: 67) in peptide fraction 17 from S15 and (reverse) primer S15R3 was designed from the amino acid sequence DDKPFYT (SEQ ID NO: 13) in peptide fraction 21 from S15. A PCR reaction with these two primers produced a 73 bp product which was sequenced to confirm it encoded a section of the S15 gene. This DNA sequence allowed the design of highly specific PCR primers S15F7 and S15R8 (FIG. 6) suitable for gene walking to obtain the flanking gene sequences.

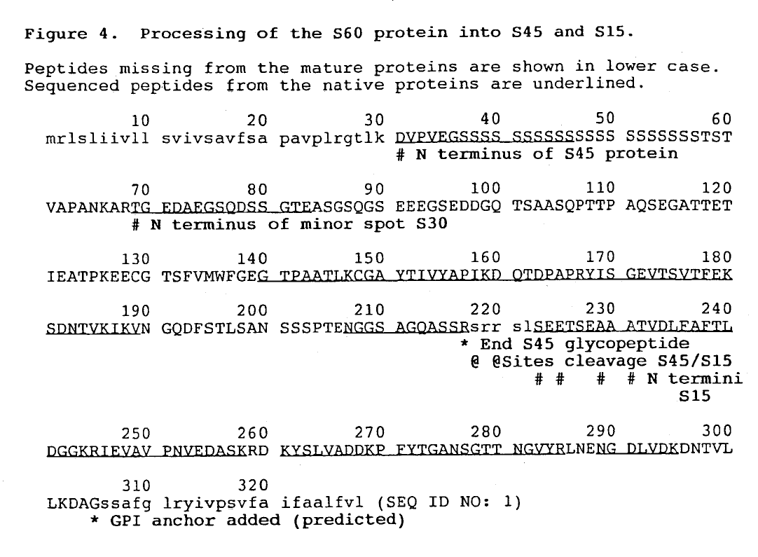

The S45 gene sequences were found immediately 5′ to the S15 gene. PCR gene walking (44) using S15R8 primer with Cryptosporidium parvum DNA digested with the restriction enzyme KpnI and ligated to the first set of gene walking primers (FIG. 6) gave the 5′ end of the S15 gene (up to 1262 bp in FIG. 2). Further gene walking using XbaI digested DNA and primer S15R10b extended the DNA sequence to the XbaI site at the start of the sequence in FIG. 2. This completed the 5′ end of the open reading frame extending beyond the start of the S15 protein. The DNA sequence encoded a long peptide 5′ to the start of S15 and in the same reading frame. The amino acid sequences obtained from S45 peptides were all present in the 5′ end of the open reading frame indicating that the two glycoproteins were produced from the same gene. In the DNA sequence (FIG. 2), the N-terminal end of S45 is close to the start of the reading frame, preceded by a consensus secretion signal with a methionine followed by a positively charged amino acid and hydrophobic region. The cleavage site between S45 and S15 can be located between the last amino acid residue (217) of peptide fraction 12 of S45 and the N-terminal sequence of S15, ie within the sequence RSRRSL (SEQ ID NO: 14) (amino acids 217-222; see FIG. 4). Note that the end of S45 is indicated by peptide fraction 12 ending at the R (217) residue, a position that would not be cut by the GluC protease. The sequence RSRR (SEQ ID NO: 9) is predicted to be very susceptible to proteolytic cleavage, especially as this protein would be exposed to trypsin during the infection process. Excysting C. parvum oocysts are known to contain both serine and cysteine protease activities (55). C. parvum oocysts also contain an amino peptidase activity (62) which is likely to be responsible for shortening the N-terminus of S15 to the observed start SEETS (SEQ ID NO: 11) and create the “ragged end” seen in the amino acid sequencing (see diagram FIG. 5).

The 3′ end of the S15 gene was difficult to isolate. Gene walking to the 3′ end of the S15 gene was initially unsuccessful, so the gene walking primers were changed to the second set shown in FIG. 6 allowing a greater range of restriction enzymes to be used. Success was achieved using NsiI digested DNA ligated to primer set 2 and using the gene specific S15F9 primer. This allowed the cloning of the flanking DNA to the NsiI site at 3121 bp in FIG. 2. The DNA sequence showed that the 3′ end of the S15 gene encodes a hydrophobic peptide as found in signal sequences for the addition of a GPI anchor. The predicted site for the addition of a GPI is at the C-terminal side of the glycine residue in the sequence KDAGSSAF (SEQ ID NO: 12). Note that a GPI anchor is required for S15 to partition into the detergent phase of the Triton X114 fractionation.

The S45 and S15 glycoproteins behave as a single membrane protein S60. The cleavage of the parent protein may occur prior to excystation since SDS-PAGE of fresh oocysts demonstrate the presence of S15 and S45 and relatively little S60. However, S45 and S15 fractionate together at all stages of the protein purification process until they are run on an SDS gel. Both peptides are found in the detergent phase of a Triton X114 fractionation; a property which is characteristic of membrane proteins, despite the absence of any hydrophobic sequences in S45. These observations could indicate that under most conditions the two peptides bind together as one protein. Note there is no cysteine in S15 so there can be no disulphide bridge between the two peptides. On isoelectric focusing, two proteins focus at the same pI despite having different pI's of 4.17 and 3.94 predicted from the amino acid sequence. However, the calculated p1 may not be accurate due to unknown modifications.

Peptides S60, S15 and S45 have been expressed as recombinant proteins in E. coli. The DNA sequences encoding the mature peptides of S60, S15 and S45 were amplified by PCR techniques using the expression oligonucleotides shown in FIG. 6. The PCR fragments were inserted into pBAD TOPO TA expression vector (Invitrogen) and expression in Top10 cells induced by the addition of 1-arabinose to the culture medium. The recombinant protein S60 was detected by a monoclonal antibody to a HIS tag (Invitrogen) and had apparent molecular weights of approximately 30 kDa. The difference in size from the 60 kDa native protein is likely to be due to lack of glycosylation and other post-translational modifications since the molecular weights predicted from the amino acid sequence are 28.2, 18.7 and 9.1 for the S60, S45 and S15 peptides respectively.

Lectins were tested by immunofluorescence on partially excysted oocysts. Lectins of H. pomatia, Helix asperse and Vicia villosa, which specifically recognize terminal a-D-N-acetylgalactosamine residues, strongly reacted with the sporozoite surface. These lectins also reacted with the inner oocyst wall, but not the outer wall on intact oocysts. H. pomatia lectin was also found to react with antigen trails shed by migrating sporozoites. Interestingly, lectin from Bandeiraea simplicifolia, which recognizes a-D-galactose and a-N-acetylgalactosamine was specific for the sporozorite surface and not oocyst walls. Lectins with specificity for mannose and glucose residues showed a weak (Lens culinaris) or no recognition (Canavalia ensiformis (Con A)) of intact oocysts, but reacted somewhat with the sporozorite surface and the inner oocyst wall.

H. pomatia lectin reacted strongly in Western blots of Triton X-114 fractionated sporozorite proteins, recognizing antigens similar in size to the major biotinylated proteins S16, S25, S30, S45, S70 and S84. These proteins are probably identical to the antigens of the invention as is indicated by their similar relative intensities and the distribution of the detected bands in the Triton X-114 fractions and also the recognition of the soluble S45 protein in the detergent depleted phase. No detectable reaction with any of the identified surface proteins was observed with lectin ConA, in contrast to an earlier study (71). However, under immunofluorescence, ConA bound to the sporozorite surface and released antigen trails. The reaction of ConA may be directed against mannose containing glycolipids exposed on the sporozoite surface and also released from the surface (72).

These data indicate that the native S60 antigen and its products most likely have terminal a-D-N-acetylgalactosamine residues which may be important in selecting appropriate expression vectors for the recombinant antigen in order to obtain optimal immunogenicity and protection against infection.

Mouse antisera raised against the recombinant S60 antigen were tested by immunofluorescence against oocyst excystation mixtures. A strong reaction with the surface of sporozoites confirmed the surface location of the S60 antigen. The antibodies also reacted with the inner wall of some empty oocyst shells, but not with intact oocysts. The labelling of the inner oocyst wall of excysted shells may be caused by the partial shedding of the S45 subunit. These observations are consistent with the surface location of the native S60 antigen and derivatives and confirm that the antigen that has been cloned is the same as the antigen that was initially purified. Furthermore, it demonstrates that the recombinant antigen expressed in E. coli is capable of eliciting antibodies in vaccinated animals that are capable of recognising the surface of excysted oocysts. This indicates that the recombinant antigen should be effective as a vaccine antigen to provide protection against infestation by Cryptosporidium.

The recombinant antigen can be expressed in a range of standard expression systems utilised in the field. These include E. coli systems that have the advantage that they are well characterised and varied and lead to the expression of the proteins at high levels. However, E. coli systems have the disadvantage that they do not glycosylate proteins which may be important for proteins such as S60 to adopt the appropriate conformation, they do not efficiently secrete proteins across a membrane so the proteins often do not fold into a native conformation and are frequently expressed intracellularly as insoluble inclusion bodies that require refolding post-purification. Thus, it is usually preferable to express such recombinant proteins in eukaryote systems and to secrete the proteins across the cell membrane into the culture supernatant in order to overcome the problems associated with the E. coli expression systems. The eukaryote systems generally lead to lower levels of expression but the secreted proteins are glycosylated, are generally appropriately refolded and, if proteins such as S60 are expressed without the predicted GPI anchor signal sequence, are located soluble in the culture supernatant which facilitates their purification. Such hosts for such expression include yeasts such as Saccharomyces cerevisiae, Pichia pastoris and Schizosaccharomyces pombe or insect cells infected with recombinant baculoviruses.

In each of these cases, the recombinant organism would be cultured, the expression of the recombinant antigen is usually induced by the addition of an appropriate compound or by a temperature shift to activate the promoter for transcription of the gene and expression is allowed to take place for between usually 1 and 24 hours. At this stage, the culture is harvested and the antigen purified using procedures that are common in the art. The degree of purity varies with the, particular application and the animal species to be treated but is rarely less than 80% and is usually greater than 95% pure.

The purified antigen is usually formulated together with a chemical referred to as an adjuvant that is active in stimulating the immune system. Commonly utilised adjuvants include aluminium hydroxide, Quil A, Saponin or oil adjuvants such as montanide marcol or Freunds adjuvants. In some cases such as in oral or inhalant applications, adjuvants are not utilised and a larger dose of antigen is required as a consequence. Formulations are prepared such that they contain between 1 and 1000 ug per dose, commonly between 20 and 100 ug per dose in cases that include an adjuvant. Vaccinations are given to the animals on a regular basis usually at least twice in the first period of risk or the first year followed by subsequent booster vaccinations during subsequent risk periods. In the case of cryptosporidiosis, to passively protect suckling animals, dams may be vaccinated prior to their first pregnancy and receive a booster vaccination during the final trimester of each pregnancy. This protocol would lead to high levels of antibodies in the colostrum and milk that would be expected to lead to effective passive protection of the suckling young. For immuno-compromised individuals who are suffering from infection by cryptosporidiosis, vaccinations may be more often and frequent at perhaps 2- to 6-month intervals in order to establish and to maintain a high degree of immune status.

Alternate expression systems that could be considered include a variety of live viruses and the injection of recombinant DNA coding for the antigen into the animal. Each of these vaccination systems is reported to stimulate different arms of the immune response.

### Uses

S60, S45 and S15: Four Applications

1. The easiest application to test clinically is to use high titre antisera to S60 to orally treat immunosuppressed patients with C. parvum diarrhoea. For example, AIDS patients die of secondary infections, frequently wasting away with diarrhoea caused by C. parvum. The only treatment known to be effective for cryptosporidosis in AIDS patents is oral administration of colostrum from hyperimmunised cows. However, this treatment is not generally available because of production difficulties, both with the purification of antigen from faeces and with the limited availability of colostrum. Treatment with oral bovine antibodies should have few problems obtaining ethical approval, being a permitted component of food. Once a treatment has been established in this desperate group of patients then the antiserum should be readily applicable to a more general group of susceptible patients including immunosuppressed patients, children and old people. Note, the market for C. parvum treatment may be quite large as at present most C. parvum infections go undiagnosed and many people may be treated prophylatically, particularly during outbreaks.

The production of high titre antisera may be done either by immunising with recombinant proteins or by direct transformation of the DNA into the animal.

2. S60 is a potential vaccine antigen, with particular application to immunodeficient patients where C. parvum infection is life threatening. Patients could be immunised before they are chemically immunosuppressed or in the case of HIV positive patients in the long period before they develop immunodeficiency. The easiest route of immunisation would be injection, but it may be desirable to boost mucosal immunity by using an inhaled or oral vaccine. Animal vaccines would also be included as possible uses. The main animal target would be household pets, probably a major source of infection for children, particularly through sandpits, grass etc contaminated with faeces. Whilst it would be possible to immunise intensively reared animals (eg calves) the utility depends on economics.

3. There is a significant demand for antigens for research into the C. Parvum infections. Although a small market, research applications are available immediately.

4. There is an urgent need for methods of distinguishing different pathotypes of C. parvum using serological or PCR techniques. In Sydney at present, a critical question is whether C. Parvum oocysts in water samples are a pathotype infectious to humans. The proteins involved in the infection process are most likely to be adapted to the requirements of individual hosts and to exhibit sequence variation due to immune selection. Thus, the gene S60 and flanking DNA are good candidates for the development of new PCR and antibody tests for distinguishing pathotypes of C. parvum.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Current, W. L. & Garcia, S. L. (1991) Clin. Microbiol. Rev. 4, 325-358.

2. Gross, T. L., Wheat, J., Bartlett, M. & O'Conner, K. W. (1986) Am. J. Gastroentrol. 81, 456.

3. Clark, D. P. & Sears, C. L. (1996) Parasitology Today 12, 221-225.

4. Casemore, D. P., Sands, R. L. & Curly, A. (1985) J. Clin. Pathol. 38, 1321.

5. Current, W. L., Reese, N. R., Ernst, J. V., Bailey, W. S., Heyman, M. B. & Weinstein, W. M. (1983) N. Engl. J. Med. 308, 1252.

6. Fayer, R., Barta, J. R., Guidry, A. & Blagburn, B. L. (1991) J. Parasitol. 77, 487-490.

7. Janoff, E. N. & Reller, L. B., (1987) J. Clin. Microbiol. 25, 967.

8. Holley, H. P. & Dover, C. (1986) J. Infect. Dis. 153, 365.

9. Heyworth, M. F. (1992) J. Infect. Dis. 166, 465-472.

10. Miller, R. A., Holmberg, R. E. & Clausen, C. R. (1983) J. Pediatr. 103, 256-259.

11. Hoepelman, A. I. M. (1996) Antimicrobial Chemotherapy 37, 871-880).

12. Stine, K. C., Harris, J. A., Linsey, N. J. & Cho, C. T. (1985) Clin. Pediatr. 24, 722-724.

13. Riggs, M. W. & Perryman, L. E. (1987) Infect. Immun. 55, 2081.

14. Fayer, R., Riggs, M. W. & Perryman, L. E. (1989) J. Parasitol. 75, 151.

15. Riggs, M. W., McGuire, T. C., Mason, P. H. & Perryman, L. E. (1989) J. Immunol. 143, 1340-1345.

16. Riggs, M. W, Cama, V. A, Leary, H. L., Sterling,C. R. (1994) Infect. Immun. 62, 1927-1939.

17. Tzipori, S. Roberton, D. & Chapman, C. (1986) Br. Med. J. 293, 1276.

18. Tzipori, S. Roberton, D., Cooper, D. A. & Chapman, C. (1987) Lancet 1, 344.

19. Fayer, R., Guidry, A. & Blagburin, B. (1990) Infect. Immun. 58, 2962-2965.

20. Ungar, B. L. P. & Nash, T. E. (1986) Infect. Immun. 53, 124-128.

21. Nord, J., Ma, P., Dijohn, D. Tzipori, S. & Tacket, C. O. (1990) AIDS (Philadelphia) 4, 581-584.

22. Perryman, L. E., Riggs, M. W., Mason, P. H. & Fayer, R. (1990) Infect. Immun. 58, 257-259.

23. Arrowood, M. J., Mead, J. R., Mahrt, J. L. & Sterling, C. R. (1989) Infect. Immun. 57, 2283-2288.

24. Fayer, R., Speer, C. A. & Dubey, J. P. (1990) In Cryptosporidiosis of man and Animals. Editors J. P. Dubey, C. A. Speer & R. Fayer. pp 31-50. CRC Press, Boca Raton.

25. Uhl, E. W., O° Connor, R. M., Perryman, L. E. & Riggs, M. W. (1992) Infect. Immun. 60, 1703-1706.

26. Nina, J. M., McDonald, V., Deer, R. M.,Wright, S. E., Dyson, D. A., Chiodini, P. L., McAdam, K. P. (1992) Parasite Immunol. 14, 227-232.

27. Mead, J. R., Arrowood, M. J. & Sterling, C. R. (1988) J. Parasitol. 74, 135-143.

28 Lumb, R., Smith, P. S., Davies, R., O'Donoghue, P. J. Atkinson, H. M. & Laiiser, J. A. (1989) Immnunol. Cell Biol. 67, 267-270.

29. Arrowood, M. J., Sterling, C. R. & Healy, M. C. (1991) J. Parsitol. 77, 315-317.

30. Tilley, M. & Upton, S. J. (i994) FENIS Nicrobiol. Letters 120, 275-278.32. Joe, A., Hamer, D. H., Kelley, M. A., Pereira, M. E. A., Keusch, G. T., Tzipori, S.,Ward, H. D. (1994) J. Euk. Microbiol. 41, S44.

31. Tilley, M., Upton, S. J., Fayer, R., Barta, J. R., Chrisp, C. E., Freed, P. S., Blagburn, B. L., Anderson, B. C. & Barnard, S. M. (1991) Infect. Immun. 59, 1002-1007.

32. Joe, A., Hamer, D. H., Kelley, M. A., Pereira, M. E. A., Keusch, G. T., Tzipori, S.,Ward, H. D. (1994) J. Euk. Microbiol. 41, S44.

33. Okhuysen, P. C., Dupont, H. L., Sterling, C. R., Chappell, C. L. (1994) Infect. Immun. 62, 4667-4670.

34. Nesterenko, M. V., Tilley, M., Upton, S. J. (1995) Microbios. 83, 77-88.

35. Jenkins, M. C, Fayer, R., Tilley, M. & Upton, S. J. (1993) Infec. Immun. 61, 2377-2382.

36. Jenkins, M., Kerr, D., Fayer, R. & Wall, R. (1995) Vaccine 13, 1658-1664.

37. Mead,J. R., Lloyd,R. M., You,X. D., Tuckerburden,C., Arrowood,M. J., Schinazi,R. F. (1994) J. Euk. Microbiol. 41, S51.

38. Perryman, L. E., Jasmer, D. P., Riggs, M. W., Bohnet, S. G., McGuire, T. C., Arrowood, M. J. (1996) A cloned gene of Cryptosporidium parvum encodes neutralization-sensitive epitopes. Mol. Biochem. Parasitol. 80, 137-147.

39. Pererva, N. (1998) PhD Thesis, submitted to the Macquarie University, Sydney

40. Bordier, C. (1981) J. Biol. Chem. 256, 1604-1607.

41. Dingermann, T., Troidl, E., Broker, M. & Nerke, K. (1991) Appl. Microbiol. Biotech. 35, 496-503.

42. Fasel, N., Begdadi-Rais, C., Bernard, M., Bron, C., Corradin, G. & Reymond, C. D. (1992) Gene 111, 157-163

43. Samyellowe, T Y (1996) Parasitol. Today 12, 308-316.

44. Morris, D. D., Reeves, R. A., Gibbs, M. D., Saul, D. J. & Bergquist, P. L. (1995) Appl. Env. Microbiol. 61, 2262-2269.

45. Wilczynska, Z. & Fisher, P. R. (1994) Plasmid 32, 182-194.

46. Petiy, F., Robinson, H. A. & McDonald, V. (1995) J. Clin. Microbiol. 33, 1922-1924.

47. Fernandez, J., Andrews, L. & Mische, S. M. (1994) Anal. Biochem. 218, 112-117.

48. Harp, J. A. & Goff, J. P. (1995) Protection of calves with a vaccine against Cryptosporidium parvum. Journal of Parasitology 81, 54-57.

49. Fayer, R., Graczyk, T. K. and Cranfeild, M. R. (1995) Multiple isolates of Cryptosporidium serpentis from captive snakes are not transmissable to neonatal BALB/c nice (Mus. musculus). Journal of Parasitol. 81, 482-484.

50. Graczyk, T. K., Fayer, R. & Cranfeild, M. R. (1996) Cryptosporidium parvum is not transissable to fish, amphibions or reptiles. Journal Parasitol. 82, 748-751.

51. Khramtov, N. V., Opert, B., Montelone, B. A. and Upton, S. J. (1997) Sequencing and expression in Escherichia coli of a gene encoding a 15 kDa Cryptosporidium parvum protein. Biochemical Biophysical Research Communications 230, 164-166.

52. Enriquez, F. J. & Riggs, M. W. (1998) Role of Immunoglobin A Monoclonal antibodies against P23 in controling murine Cryptosporidium parvum infection. Infection and Immunity 66, 4469-4473.

53. Ungar, B. I. P., Ward, D. J., Fayer, R. & Quinn, C. A. (1990) Cessation of Cryptosporidium associated diarrhoea in an acquired immunodeficency syndrome patient after treatment with hyperimmune bovine colostrum. Gastroenterology 98, 486-489.

54. Perryman, L. E., Kegerreis, K. A. & Mason, P. H. (1993) Effect of orally administered monoclonal antibody on persistent Cryptosporidium parvum infection in scid mice. Infection and Immunity, 61, 4906-4908.

55. Forney, J. R., Yang, S.G. & Healey, M. C. (1996) Protease activity associated with excistation of Cryptosporidium parvum oocysts. Journal of Parasitology 82, 889-892.

56. Laemmli, U. K. (1970) Nature (London) 227, 680-685

57. Khyse-Anderson, J. (1984) Biochemical and Biophysical Methods 10, 203-209

58. Fernandez, J., Andrews, L., and Sheenah, M. (1994) Analytical Biochemistry 218, 112-117

59. Brune, D. C. (1992) Analytical Biochemistry 207, 285-290

60. Carson, W., and Stone, S. (1995) In Sequence, Beckman protein sequencing newsletter 3, 1-9

61. Bordier, C. (1981) Journal of Biological Chemistry 256, 1604-1607

62. Okhuysen, P. C., DuPont, H. L., Sterling, C. R. & Chappell, C. L. (1994) Arginine aminopeptidase, an integral membrane protein of the Cryptosporidium parvum sporozoite. Infection Immunity 62, 4667-4670.

63. MacKenzie et al, (1994) A massive outbreak in Milwaukee of Cryptosporidium infection transmitted through the public water supply. New England Journal of Medicine 331, 161-167.

64. Fayer, R. Cryptosporidium and Cryptosporidosis. CRC Press, Boca Raton, USA.

65. Current, W. L. (1994) Cryptosporidium parvum: household transmission. Annals of Internal Medicine 120, 518-519.

66. Peng, M. M. et al. (1997) Genetic polymorphism among Cryptosporidium parvum isolates: evidence of two distinct transmission cycles. Emerging Infectious Diseases 3, 567-573.

67. Spano, F., Putignani, L., Mclauchlin, J., Casemore, D. P. & Crisanti, A. (1997) PCR-RFLP analysis of the Cryptosporidium oocyst wall protein (COWP) gene descriminates between C. wrairi and C. parvum, and between C. parvum isolates of human and animal origin. FEMS Microbiology Letters 150, 209-217.

68. Jung, E., Gooley, A. A., Packer, N. H., Karuso, P. & Williams, K. L. (1998) Rules for the addition of N-linked N-acetyl glucosamine to secreted proteins in Dictyostelium discoideum: In vivo studies on glycosylation of mucin MUC1 and MUC2 repeats. European Journal of Biochemistry 253, 517-524.

69. Sagodira S, Buzoni-Gatel D, Iochmann S, Nachiri M, Bout D. (1999) Protection of kids agains Cryptosporidium parvum infection after immunisation of dams with CP15-DNA. Vaccine, 17: 2346-2355.

70. Iochmann S, Sagodira S, Nievelec M, Reperant J, Naciri M, Coursaget P, Bout D (1999) Comparison of the humoral and cellular responses of two proparations of Cryptosporidium parvum CP15/60 recombinant protein. Microbial. Pathogenesis. 26: 307-315

71. Tilley M, Upton S J (1990) Electrophoretic characterization of Cryptosporidium parvum (KSU-1 isolate) (Apicomplexa: Cryptosporiidae). Can J. Zool. 68:1513-1519

72. Riggs M W, McNeil M R, Perrymann L E, Stone A L, Sherman M S, O'Connor R M (1999) Cryptosporidium parvum sporozorite pellicle antigen recognized by a neutralising monoclonal antibody is a b-mannosylated glycolipid. Infect. Immun. 67:1317-1322

73. Pedraza-Diaz S, Amar C, McLauchlin, J (2000) The identification and characterisation of an unusual genotype of Cryptosporidium from human faeces as Cryptosporidium meleagridis , FEMS Microbiology Letters, 189 : 189-194

74. Sreter T, Kovacs G, Da Silva A. J, Pieniazek N. J, Szell Z, Dobos-Kovacs M, Marialigeti K, Varga I. (2000) Morphologic, host specificity, and molecular characterization of a Hungarian Cryptosporidium meleagridis isolate. Applied and Environmental Microbiology, 66: 735-738

1. An isolated nucleic acid molecule encoding a Cryptosporidium polypeptide comprising the amino acid sequence:
MRLSLIIVLLSVIVSAVFSAPAVPLRGTLKDVPVEGSSSSSSSSSSSSSS SSSSSSSTSTVAPANKARTGEDAEGSQDSSGTEASGSQGSEEEGSEDDGQ TSAASQPTTPAQSEGATTETIEATPKEECGTSFVMWFGEGTPAATLKCGA YTIVYAPIKDQTDPAPRYISGEVTSVTFEKSDNTVKIKVNGQDFSTLSAN SSSPTENGGSAGQASSRSRRSLSEETSEAAATVDLFAFTLDGGKRIEVAV PNVEDASKRDKYSLVADDKPFYTGANSGTTNGVIRLNENGDLVDKDNTVL LKDAGSSAFGLRYIVPSVFAIFAALFVL (SEQ ID NO: 1), a functionally equivalent sequence thereof, or part thereof having at least five amino acids.

2. A nucleic acid molecule according to claim 1, wherein the polypeptide has an amino acid sequence as shown as SEQ ID NO: 1.

3. A nucleic acid molecule according to claim 1, which comprises a nucleotide sequence substantially as shown as SEQ ID NO: 2, or a functionally equivalent nucleotide sequence thereof.

4. A nucleic acid molecule according to claim 1, which comprises a nucleotide sequence which hybridises to the nucleotide sequence of SEQ ID NO: 2.

5. A nucleic acid molecule according to claim 1, which comprises a nucleotide sequence which shows at least 60% homology with the nucleotide sequence of SEQ ID NO: 2.

6. A nucleic acid molecule according to claim 5, which comprises a nucleotide sequence which shows at least 80% homology with the nucleotide sequence of SEQ ID NO: 2.

7. A nucleic acid molecule according to claim 5, which comprises a nucleotide sequence which shows at least 90% homology with the nucleotide sequence of SEQ ID NO: 2.

8. An expression vector comprising a nucleic acid molecule according to any one of claims 1-7.

9. A host cell transformed with a nucleic acid molecule according to any one of claims 1-7 or an expression vector according to claim 8.

10. An isolated polypeptide from Cryptosporidium comprising the following sequence:
DVPVEGSSSSSSSSSSSSSSSSSSSSSTSTVAPANKARTGEDAEGSQDSS GTEASGSQGSEEEGSEDDGQTSAASQPTTPAQSEGATTETIEATPKEECG TSFVMWFGEGTPAATLKCGAYTrVYAPIKDQTDPAPRYISGEVTSVTFEK SDNTVKIKVNGQDFSTLSANSSSPTENGGSAGQASSRSRRSL SEETSEAAATVDLFAFTLDGGKRIEVAVPNVEDASKRDKYSLVADDKPFYT GAiNSGTTNGVYRLNENGDLVDKDNTVLLKDAG (SEQ ID NO: 3), or a functionally equivalent sequence thereof, or part thereof having at least five amino acids.

11. A polypeptide according to claim 10, wherein the polypeptide has the amino acid sequence shown as SEQ ID NO: 1.

12. A polypeptide according to claim 10, wherein the polypeptide has the amino acid sequence:
DVPVEGSSSSSSSSSSSSSSSSSSSSSTSTVAPANKARTGEDAEGSQDSS GTEASGSQGSEEEGSEDDGQTSAASQPTTPAQSEGATTETEEATPKEECG TSFVMVFGEGTPAATLKCGAYTIVYAPIKDQTDPAPRYISGEVTSVTFEK SDNTVKIKVNGQDFSTLSANSSSPTENGGSAGQASSRSRRSL SEETSEAAATVDLFAFTLDGGKRIEVAVPNVEDASKRDKYSLVADDKPFYT GANSGTTNGVYRLNENGDLVDKDNTVLLKDAG (SEQ ID NO: 3), wherein at least the underlined amino acids have been modified by a reducing terminal α-GalNAc, or a functionally equivalent sequence thereof, or part thereof having at least 5 amino acids.

13. An isolated polypeptide from Cryptosporidium comprising one of the following sequences:
SEETSEAAATVDLFAFTLDGGKRIEVAVPNVEDASKRDKYSLVADDKPFYT GANSGTTNGVYRLNENGDLVDKDNTVLLKDAG (SEQ ID NO: 7); ETSEAAATVDLFAFTLDGGKRIEVAVPNVEDASKRDKYSLVADDKPFYT GANSGTTNGVYRLNENGDLVDKDNTVLLKDAG (SEQ ID NO: 64); AAATVDLFAFTLDGGKRIEVAVPNVEDASKRDKYSLVADDKPFYT GANSGTFNGVYRLNENGDLVDKDNTIVLLKDAG (SEQ ID NO: 65); and ATVDLFAFTLDGGKRIEVAVPNVEDASKRDKYSLVADDKPFYT GANSGTTNGVYRLNENGDLVDKDNTVLLKDAG (SEQ ID NO: 66), or a functionally equivalent sequence thereof, or part thereof having at least five amino acids.

14. A polypeptide according to claim 13, wherein the polypeptide has the amino acid sequence shown as SEQ ID NO: 6, SEQ ID NO: 64, SEQ ID NO: 65 or SEQ ID NO: 66.

15. A vaccine preparation against Cryptosporidium containing one or more polypeptides comprising an amino acid sequence selected from:
(SEQ ID NO: 4)
DVPVEGSSSSSSSSSSSSSSSSSSSSSTSTVAPANIKARTGEDAEGSQDS
SGTEASGSQGSEEEGSEDDGQTSAASQPTTPAQSEGNPTETIEATPKEEC
GTSFVMWFGEGTPAATLKCGAYTIVYAPIKDQTDPAPRYISGEVTSVTFE
KSDNTVKIKVNGQDFSTLSANSSSPTENGGSAGQASSR;
or functionally equivalent sequences thereof, or parts thereof having at least five amino acids.

16. A vaccine preparation against Cryptosporidium containing one or more polypeptides comprising an amino acid sequence selected from:
(SEQ UD NO: 5)
TGEDAEGSQDSS;
(SEQ ID NO: 6)
GTEASGSQGSEEEGSEDDGQTSAASQPTTPAQSEGATTETIEATPKEECG
TSFVMWFGEGTPAATLKCGAYTIVYAPIKDQTDPAPRYISGEVTSVTFEK
SDNTVKIKVNGQDFSTLSANSSSPTENGGSAGQASSRSR;
or functionally equivalent sequences thereof, or parts thereof having at least five amino acids.

17. A vaccine preparation against Cryptosporidium containing one or more polypeptides comprising an amino acid sequence selected from:
(SEQ ID NO: 7)
SEETSEAAATVDLFAFTLDGGKRIEVAVPNVEDASKRDKYSLVADDKPFYT
GANSGTTNGVYRLNENGDLVDKDNTVLLKDAG;
or functionally equivalent sequences thereof, or parts thereof having at least five amino acids.

18. A method of immunising a subject against Cryptosporidium, the method comprising providing a vaccine preparation according to any one of claims 15-17 to a subject such that an immune response is generated in the subject against Cryptosporidium.

19. A method according to claim 17, wherein said subject is a human.
